(19) United States
(12) Patent Application Publication
(10) Pub. No.: US 2010/0310532 A1
(43) Pub. Date: Dec. 9, 2010

(54) GENE TARGETS IN ANTI-AGING THERAPY AND TISSUE REPAIR

(75) Inventors: Joshua M. Hare, Miami Beach, FL (US); Bettina Heidecker, Miami, FL (US)

Correspondence Address: NOVAK DRUCE + QUIGG LLP (WPB), 525 Okeechobee Blvd, 15th Floor, City Place Tower, West Palm Beach, FL, 33401, US

(73) Assignee: University of Miami, Miami, FL (US)

(21) Appl. No.: 12/676,003

(22) PCT Filed: Sep. 8, 2008
(86) PCT No.: PCT/US2008/075611

§ 371 Date: Aug. 12, 2010

Related U.S. Application Data
(60) Provisional application No. 60/970,385, filed on Sep. 6, 2007.

(51) Int. Cl. A61K 31/7088; C12Q 1/68; C07K 16/18; C12N 5/10; C12N 15/63; A61K 38/17; A61K 38/43; A61K 35/12; C12Q 1/02; G01N 33/53; C40B 30/04; A61P 9/00

(52) U.S. Cl. 424/93.21; 435/6; 530/389.1; 435/325; 435/372; 435/366; 435/320.1; 514/1.1; 514/44 R; 424/94.1; 435/29; 435/7.1; 506/9; 435/375

(57) ABSTRACT

Novel gene and protein targets are described for treatment of cardiac disorders, anti-aging therapies and tissue repair. Identified biomarkers are prognostic of long term survival of patients suffering from heart diseases and related disorders.

### CROSS-REFERENCE TO RELATED APPLICATIONS

The present application claims the priority of U.S. provisional patent application No. 60/970,385 entitled “GENE TARGETS IN ANTI-AGING THERAPY AND TISSUE REPAIR” filed Sep. 6, 2007, which is incorporated herein by reference in its entirety.

### STATEMENT REGARDING FEDERALLY SPONSORED RESEARCH

This invention was made with U.S. government support under grant numbers M400-217-2954 and RO-1 HL-65455 both awarded by the National Institutes of Health. The U.S. government has certain rights in the invention.

### FIELD OF THE INVENTION

This invention relates to target genes in anti-aging therapy, tissue repair, treatment of cardiac disorders and stem cell viability.

### BACKGROUND

The current approach to the treatment of patients with idiopathic cardiomyopathy and heart failure lacks individualization and becomes increasingly important in the future, as the number of classes of medicine for heart failure increase. While substantial research has demonstrated the prognostic value of a variety of clinical characteristics in heart failure, the ability to distinguish cardiomyopathy patients who will improve their ejection fraction and functional status from those who will go on to develop circulatory collapse and require cardiac transplantation or VAD placement is still not possible.

### SUMMARY

This Summary is provided to present a summary of the invention to briefly indicate the nature and substance of the invention. It is submitted with the understanding that it will not be used to interpret or limit the scope or meaning of the claims.

Embodiments of this invention describe a novel biomarker comprising Rad50, Smg6, Mre11 and the Mre11/Rad50/Nbs1 (MRN) complex. This biomarker provides a prognostic tool for heart failure and other cardiac diseases, is the base for individualizing treatment, and identification of new therapies. These therapeutic target genes, were shown to participate and enhance telomere activity and genes were observed to play a fundamental role in stem cell viability. The approaches utilized herein, have applications for anti-aging therapies and tissue repair.

In a preferred embodiment, a biomarker composition prognostic for the long term survival of patients recovering from heart failure and cardiac diseases comprises Smg6 and Rad50 markers and polynucleotide and polypeptide molecules thereof. In another embodiment, the biomarker further comprises Mr11 and any other marker of the Mre11/Rad50/Nbs1 (MRN) complex.

In another preferred embodiment, markers Smg6, Rad50, Mre11, Mre11/Rad50/Nbs1 complex are detected in cells, preferably mammalian cells. For example, stem cell, monocyte, lymphocyte, neutrophil, eosinophil, mast cell, natural killer cells, myocytes, heart cells and the like. The cells can be obtained from a patient (patient derived), a normal individual, xenogeneic, allogeneic, cell lines and the like.

In another preferred embodiment, the biomarker further comprises a marker selected from the group consisting of Mre11, Rad50, Nbs1, Mre11/Rad50/Nbs1 complex, enzymes and factors involved in the functions of the complex such as for example, nucleases, topoisomerases, telomerases, Rec, Sir, etc and t-loop generation factors.

In one preferred embodiment, the biomarker comprises the Rad50, Smg6, and Mre11 markers and at least one marker comprising a polynucleotide, fragments, alleles, variants, mutants, complementary sequences thereof of Rad50, Smg6, and Mre11 and combinations thereof.

In another embodiment, the biomarker further comprises the Rad50, Smg6, and Mre11 markers and at least one marker comprising a polypeptide, fragment, variants, analogs and mutants thereof of Rad50, Smg6, and Mre11 and combinations thereof.

In another preferred embodiment, the biomarker further comprises the Rad50, Smg6, and Mre11 markers and at least one marker comprising a polynucleotide, fragments, alleles, variants, mutants substituted polynucleotides and complementary sequences thereof of Smg6.

In another preferred embodiment, the biomarker further comprises the Rad50, Smg6, and Mre11 markers and at least one marker comprising a polypeptide, fragment, variants, analogs and mutants thereof of Smg6.

In another preferred embodiment, the biomarker further comprises the Rad50, Smg6, and Mre11 markers and at least one marker comprising a polynucleotide, fragments, alleles, variants, mutants, substituted polynucleotides and complementary sequences thereof of Rad50.

In another preferred embodiment, the biomarker further comprises the Rad50, Smg6, and Mre11 markers and at least one marker comprising a polypeptide, fragment, variants, analogs and mutants thereof of Rad50.

In another preferred embodiment, an agent specifically binds a biomarker composition comprising Smg6, Rad50, Mre11, Nbs1, Mre11/Rad50/Nbs1 complex, enzymes and t-loop generation factors, variants, mutants, and fragments thereof, wherein said agent comprises an antibody, aptamer or complementary polynucleotides which hybridize under stringent hybridization conditions to Smg6, Rad50, Mre11, Nbs1, Mre11/Rad50/Nbs1 complex, enzymes and t-loop generation factors, variants, mutants, and fragments thereof.

In another preferred embodiment, an agent modulates the expression of Rad50, Smg6, and Mre11 and the MRN complex when a cell is exposed to said agent.

In another preferred embodiment, an isolated cell expresses Smg6 and/or Rad50 and/or Mre11/Rad50/Nbs1 complex, enzymes and t-loop generation factors, variants, mutants, and fragments thereof. In one aspect the cell is a mammalian stem cell. In other embodiments, the cells comprise, circulating cells such as for example, monocytes, lymphocytes, macrophages, natural killer cells; cardiac cells, myocytes, cells involved in tissue repair, remodeling, maintenance and the like.

In another preferred embodiment, the isolated cell comprises a vector expressing Smg6 and/or Rad50 and/or Mre11, and/or Nbs1, and/or the Mre11/Rad50/Nbs1 complex, enzymes and t-loop generation factors, variants, mutants, and fragments thereof.

In another preferred embodiment, the Smg6, Rad50, Mre11, Nbs1, Mre11/Rad50/Nbs1 complex, enzymes and t-loop generation factors variants, mutants and fragments thereof are operatively linked to a tissue specific promoter, a constitutive or inducible promoter.

In another preferred embodiment, a vector expresses Smg6 and/or Rad50, and/or the Mre11/Rad50/Nbs1 complex, enzymes and t-loop generation factors, variants, mutants, and fragments thereof, and are operably linked to a tissue-specific promoter, a constitutive or inducible promoter.

In another preferred embodiment, a method of treating heart failure comprises administering to a patient a composition comprising Rad50, Smg6, Mre11, Nbs1, or the Mre11/Rad50/Nbs1 complex, enzymes and t-loop generation factors, variants, mutants, and fragments thereof. In one aspect, the Smg6, Rad50, Mre11, Nbs1 and the Mre11/Rad50/Nbs1 complex comprise polynucleotides and/or polypeptides.

In another aspect, treatment comprises administering an agent to a patient which induces the expression of Rad50.

In another aspect, treatment comprises administering an agent to a patient which induces the expression of Mre11.

In another aspect, treatment comprises administering an agent to a patient which induces the expression of Smg6.

In another preferred embodiment, the method of treating heart failure comprises a composition comprising an expression vector encoding Smg6, Rad50, Mre11, Nbs1 or the Mre11/Rad50/Nbs1 complex operably linked to a promoter. The promoter comprises a tissue specific promoter, a constitutive tissue promoter or an inducible tissue specific promoter.

In another preferred embodiment, cells are treated with an agent which increases expression of Rad50, Smg6, Mre11, Nbs1, or the Mre11/Rad50/Nbs1 complex.

In another preferred embodiment, a method of treating heart failure comprises isolating stem cells from a patient or donor; administering to the stem cells a composition comprising Rad50, Smg6, Mre11, Nbs1, or the Mre11/Rad50/Nbs1 complex, enzymes and t-loop generation factors, variants, mutants, and fragments thereof; culturing the cells ex-vivo; administering the cells to a patient; and, treating heart failure.

In another preferred embodiment, the cells comprise, circulating blood cells, cells involved in tissue repair and remodeling. The cells can be combinations of cells, for example, stem cells and any other type of cell or agents. These agents can include cellular factors such as cytokines, metalloproteinases, growth factors and the like.

In another preferred embodiment, the cells, e.g. stem cells over-express Rad50, Smg6, Mre11, Nbs1, Mre11/Rad50/Nbs1 complex, enzymes and t-loop generation factors by at least about 10% as compared to a normal cell. In one aspect, the stem cells preferably over express Rad50, Mre11, and Smg6. In another aspect, the stem cells over-express Rad50. In another aspect, the stem cells over-express Smg6. In yet another aspect, the stem cells over-express at least one polynucleotide and/or polypeptide from the Mre11/Rad50/Nbs1 complex (MRN), enzymes and factors involved in the pathways or functions in which the complex is involved in, and t-loop generation factors. Examples of MRN complex functions include DNA binding, exonuclease and endonuclease activities, DNA unwinding, and DNA end recognition. In addition to the repair processes listed above, which are mostly dependent upon homologous recombination, the MRN complex also facilitates double strand break repair via nonhomologous end joining as well as introduction of double strand breaks in meiosis, detection of damaged DNA, DNA damage checkpoint activation, telomerase recruitment, and suppression of gross chromosomal rearrangements.

In one preferred embodiment, a method of treating myocardial infarction and cardiac disorders, comprises isolating stem cells from a patient or donor; transforming the isolated stem cells with a vector expressing Rad50, Smg6, Mre11, Nbs1, or the Mre11/Rad50/Nbs1 complex, enzymes and t-loop generation factors variants, mutants, and fragments thereof, as compared to a normal cell, and/or incubating the stem cells with a pharmaceutical agent which increases the expression of Rad50, Smg6, Mre11, variants, mutants, fragments and combinations thereof as compared to a normal cell; administering the cells to a patient; and, treating myocardial infarction and cardiac disorders.

In another preferred embodiment, a method of treating heart failure, said method comprising: administering to a patient a pharmaceutical composition comprising nucleic acids and/or peptides of Rad50, Smg6, Mre11, Nbs1, or the Mre11/Rad50/Nbs1 complex, enzymes and t-loop generation factors, variants, mutants, fragments, and combinations thereof; and, treating heart failure.

In another preferred embodiment, a method of treating myocardial infarction and cardiac disorders, said method comprising: administering to a patient a pharmaceutical composition comprising nucleic acids and/or peptides of Rad50, Smg6, Mre11, Nbs1, or the Mre11/Rad50/Nbs1 complex, enzymes and t-loop generation factors, variants, mutants, fragments, and combinations thereof; and, treating myocardial infarction and cardiac disorders. In one embodiment, the Smg6, Rad50, Mre11, Nbs1, and the Mre11/Rad50/Nbs1 complex, enzymes and t-loop generation factors are upregulated as compared to a normal or diseased cell.

In another preferred embodiment, a method of selecting cells to treat a patient in need of stem cell implantation, said method comprising: isolating a stem cell; screening the stem cell for expression of Rad50, Smg6, Mre11, Nbs1, or the Mre11/Rad50/Nbs1 complex; separating and enriching the Rad50, Smg6, Mre11, Nbs1, or the Mre11/Rad50/Nbs1 complex expressing stem cells; and, selecting said stem cells to treat a patient in need of stem cell implantation. The screening of the cells, comprises detecting Rad50, Smg6, Mre11, Nbs1, or the Mre11/Rad50/Nbs1 complex expression by identifying Rad50, Smg6, Mre11, Nbs1, or the Mre11/Rad50/Nbs1 complex polynucleotides and polypeptides. The identification of either polynucleotides or polypeptides would constitute a positive selection. Examples of polynucleotides include detection of cDNA, RNA, mRNA.

In one preferred embodiment, the stem cells are autologous or donor derived.

In another preferred embodiment, the stem cells are cultured with agents which increase expression of Rad50, Smg6, Mre11, Nbs1, or the Mre11/Rad50/Nbs1 complex.

In another preferred embodiment, the stem cells are transformed with a vector expressing Rad50, Smg6 or Mre11. The cells can then be administered to a patient via a variety of routes, for example, intra cardially, intra-peritoneally, intra muscularly, intra-venously and the like.

In another preferred embodiment, a pharmaceutical composition comprises nucleic acids and/or peptides of Rad50, Smg6, Mre11, Nbs1, or the Mre11/Rad50/Nbs1 complex, variants, mutants, and fragments thereof in a therapeutically effective concentration.

In another preferred embodiment, a method of identifying candidate agents for modulating expression of Rad50, Smg6, Mre11, Nbs1, or the Mre11/Rad50/Nbs1 complex comprises providing a biological sample; incubating the biological sample with a candidate agent; screening the biological sample for expression of Smg6, Rad50, Mre11, Nbs11 and/or Mre11/Rad50/Nbs1 complex, enzymes or factors involved in the generation of t-loops; comparing the expression levels between the biological sample and control; and, identifying an inhibitor. Preferably, the Smg6, Rad50, Mre11, Nbs1 expression levels are upregulated in the biological sample contacted with a candidate agent as compared to a control sample.

In one embodiment, Rad50 expression levels are determined by measuring the amount of at least one of Rad50 polypeptide, Rad50 mRNA, and Rad50 cDNA.

In another embodiment, the expression levels of Smg6 are determined by measuring the amount of at least one of Smg6 polypeptide, Smg6 mRNA, and Smg6 cDNA.

In another preferred embodiment, the expression levels of Mre11 are determined by measuring the amount of at least one of Mre11 polypeptide, Mre11 mRNA, and Mre11 cDNA.

In another preferred embodiment, the expression levels of Rad50, Smg6 and Mre11 are determined by detecting the amount of a transcribed polynucleotide, e.g. mRNA, cDNA, or portion thereof. The method of detection further comprises amplifying the transcribed polynucleotide or portion thereof.

In another preferred embodiment, the method of identifying candidate therapeutic agents for modulating expression of Smg6 and/or Rad50 and/or Mre11 comprises providing a biological sample; incubating the biological sample with a candidate therapeutic agent; screening the biological sample for expression of Smg6, Rad50, Mre11 and/or Mre11/Rad50/Nbs1 complex, enzymes or factors involved in the generation of t-loops; comparing the expression levels between the biological sample and control; and, identifying a candidate therapeutic agent.

In another embodiment, the biological sample comprises fluid, a cell, tissues, protein, peptides, amino acids, and nucleic acids. The cells can be cell lines, transformed cells, normal cells, stem cells and the like.

In another preferred embodiment, the method of identifying candidate therapeutic agents that modulate expression of Rad50, Smg6, Mre11, and/or Mre11/Rad50/Nbs1 complex, enzymes or factors involved in the generation of t-loops, are identified by comparing expression levels in samples in the presence of the candidate therapeutic agents as compared to control cells in the absence of candidate therapeutic agents. The expression of these molecules is detected, for example, by immunoassay, SDS-PAGE gel, blotting, PCR or biochip arrays, and the like. The biochips arrays are protein chip arrays, nucleic acid arrays. One of ordinary skill in the art would be well-versed in identifying other applicable methods.

In another preferred embodiment, a method of treating heart failure comprises isolating cells from a patient or donor; screening the cells for expression of Rad50, Smg6, Mre11 or Mre11/Rad50/Nbs1 complex, enzymes and t-loop generation factors, variants, mutants, and fragments thereof; culturing the cells ex-vivo; administering the cells to a patient; and, treating heart failure. Preferably, the cells express Rad50, Smg6 and Mre11 and/or Rad50, Smg6 and Mre11 polynucleotides are identified, e.g. RNA. The cells can be from any source, for example, cell lines, autologous, heterologous, syngeneic, or allogeneic cells.

In another preferred embodiment, the cells express at least one polynucleotide and/or polypeptide from the Mre11/Rad50/Nbs1 complex, enzymes and t-loop generation factors.

In another preferred embodiment, the step of culturing the cells further comprising administering agents which increase expression of at least one of Rad50, Smg6 or Mre11.

In another preferred embodiment, a method of treating and reducing cardiovascular aging comprises administering to a patient a pharmaceutical composition comprising nucleic acids and/or peptides of Rad50, Smg6, Mre11, variants, mutants, and fragments thereof, and/or cells comprising Rad50, Smg6, Mre11, variants, mutants, and fragments thereof; and, treating and reducing cardiovascular aging.

In another preferred embodiment, a method of repairing and maintaining telomeres and modulating telomere activity comprises administering to a cell nucleic acids and/or peptides comprising Smg6, Rad50, Mre11, variants, mutants, and fragments thereof, and/or cells comprising Smg6, Rad50, Mre11, variants, mutants, and fragments thereof expressing; and, repairing and maintaining telomeres and modulating telomere activity.

In another preferred embodiment, a kit comprises a biomarker comprising at least one of: Rad50, Smg6, Mre11, Nbs1, or Mre11/Rad50/Nbs1 complex. In one embodiment, the kit further comprises an agent for detection of at least one of Rad50, Smg6, Mre11, Nbs1, or the Mre11/Rad50/Nbs1 complex.

In another preferred embodiment, the agent comprises an antibody, aptamer, oligonucleotide probe, polynucleotide, or polypeptide.

Other aspects of the invention are described infra.

### BRIEF DESCRIPTION OF THE DRAWINGS

FIG. 1 is a Kaplan Meier curve illustrating event-free survival of patients in our study population: While patients with a poor prognosis (BP) experienced an event (death, insertion of LVAD or cardiac transplant) within the first 2 years after presentation of heart failure, patients with good prognosis (GP) survived more than 5 years without any supporting device.

FIG. 2 is a SAM plot of significantly upregulated genes in patients with a good prognosis: a) 46 significantly upregulated genes in samples from patients with GP vs BP (q<5%, FC>1.2, n=30).

FIG. 3 is a graph showing average delta CT (avg dCT) values for Rad50 mRNA expression levels in different areas of the infarcted heart. RAD50 was overexpressed in the infarct zone (IZ) vs remote zone (RZ) (FC=2.8, P=0.045). *P=0.045: remote zone vs infarct zone, n=6 ea.; error bars=SEM; statistic: Holm Sidak-method)

FIG. 4 is a graph showing average delta CT values (avg dCT) of RAD50 mRNA expression levels in peripheral blood cells at baseline and 20 minutes post myocardial infarction (MI). * P=0.04; n=4 ea; error bars=SEM; statistic: student t-test.

### DETAILED DESCRIPTION

It should be understood that numerous specific details, relationships, and methods are set forth to provide a full understanding of the invention. One having ordinary skill in the relevant art, however, will readily recognize that the invention can be practiced without one or more of the specific details or with other methods. In other instances, well-known structures or operations are not shown in detail to avoid obscuring the invention. The present invention is not limited by the illustrated ordering of acts or events, as some acts may occur in different orders and/or concurrently with other acts or events. Furthermore, not all illustrated acts or events are required to implement a methodology in accordance with the present invention.

Embodiments of the invention comprise novel therapeutic target genes, Rad50, Smg6, Mre11, Nbs1, or the Mre11/Rad50/Nbs1 complex (MRN), which participate in and enhance telomere activity. These genes were observed to play a fundamental role in stem cell viability. These genes were upregulated in patients diagnosed with heart failure and idiopathic cardiomyopathy, but who recovered and survived long-term (>5 years after presentation) relative to those who died within 2 years. This long term survival was associated with functional recovery of the heart.

### DEFINITIONS

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the molecules: “Rad50”, “Smg6”, “Mre11”, “Nbs1”, or the “Mre11/Rad50/Nbs1 complex” refers to both the polynucleotide and polypeptide sequences unless described otherwise. The terms also encompasses mutants, variants, complementary sequences, analogs, fragments, etc., of the polynucleotide and/or polypeptide sequences of these molecules.

As used herein, “cardiac disorders” or “heart disease” include cardiac or cardiovascular diseases. Examples of cardiac disorders include, but are not limited to, cardiac arrhythmia, myocardial ischemia, myocardial infarction, congestive heart failure, dilated and hypertrophic cardiomyopathy, cardiac hypertrophy, cardiac transplantation and rejection, allograft rejection (cardiac), coronary angioplasty, cardiopulmonary by-pass surgery and electrophysiological studies.

The term “complement” used herein means one strand of a double-stranded nucleic acid, in which all the bases are able to form base pairs with a sequence of bases in another strand. Also, complementary is defined as not only those completely matching within a continuous region of at least 15 contiguous nucleotides, but also those having identity of at least 50%, preferably 70%, more preferably 80%, still more preferably 90%, and most preferably 95% or higher within that region.

The term “progenitor cell” is used synonymously with “stem cell.” Hence, a neural progenitor cell is a neural stem cell. Both terms refer to an undifferentiated cell which is capable of proliferation and giving rise to more progenitor cells having the ability to generate a large number of mother cells that can in turn give rise to differentiated, or differentiable daughter cells.

In a preferred embodiment, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors.

In the context of cell ontogeny, the adjective “differentiated” is a relative term. A “differentiated cell” is a cell that has progressed further down the developmental pathway than the cell it is being compared with. Thus, pluripotent embryonic stem cells can differentiate to lineage-restricted precursor cells, such as hematopoietic cells, which are pluripotent for blood cell types; hepatocyte progenitors, which are pluripotent for hepatocytes; and various types of neural progenitors listed above. These in turn can be differentiated further to other types of precursor cells further down the pathway, or to an end-stage differentiated cell, which plays a characteristic role in a certain tissue type, and may or may not retain the capacity to proliferate further. Neurons, astrocytes, and oligodendrocytes are all examples of terminally differentiated cells.

The terms “amino acid” or “amino acid sequence” refer to an oligopeptide, peptide, polypeptide, or protein sequence, or a fragment of any of these, and to naturally occurring or synthetic molecules. In this context, “fragments,” or “portions” refer to fragments of protein which are preferably at least 10 to about 30 or 50, 60, 70, 80 90, 100, 200 or 300 amino acids in length, preferably at least 15, 20, 25, 30, 40, or 50 amino acids. Representative examples of peptide or polypeptide fragments are 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids in length. Where “amino acid sequence” is recited to refer to an amino acid sequence of a naturally occurring protein molecule, “amino acid sequence” and like terms are not meant to limit the amino acid sequence to the complete native amino acid sequence associated with the recited protein molecule.

As referred to herein, “fragments of a nucleic acid sequence” or “portions of a nucleic acid sequence” or “portion” comprise at least about 10 or 15 nucleic acid residues (nucleotides), or at least about 20, 30, 40, 50, 60, 70, 80, 90, 100, 150 or 200 nucleic acid residues. Representative examples of oligonucleotide fragments are 2, 3, 4, 5, 6, 7, 8, 9, 10, 11, 12, 13, 14, 15, 16, 17, 18, 19 or 20 nucleotides in length.

As used herein, “polynucleotide” refers to a chain of at least two nucleic acid monomers which can be deoxyribonucleic acids, ribonucleic acids, natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. The polynucleotide may be “chimeric,” that is, composed of different regions. In the context of this invention “chimeric” compounds are polynucleotides, which contain two or more chemical regions, for example, DNA region(s), RNA region(s), PNA region(s) etc.

As used herein, the term “monomers” typically indicates monomers linked by phosphodiester bonds or analogs thereof to form polynucleotides ranging in size from a few monomeric units, e.g., from about 3-4, to about several hundreds of monomeric units. Analogs of phosphodiester linkages include: phosphorothioate, phosphorodithioate, methylphosphornates, phosphoroselenoate, phosphoramidate, and the like, as more fully described below.

In the present context, the terms “nucleobase” covers naturally occurring nucleobases as well as non-naturally occurring nucleobases. It should be clear to the person skilled in the art that various nucleobases which previously have been considered “non-naturally occurring” have subsequently been found in nature. Thus, “nucleobase” includes not only the known purine and pyrimidine heterocycles, but also heterocyclic analogues and tautomers thereof. Illustrative examples of nucleobases are adenine, guanine, thymine, cytosine, uracil, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)-alkynylcytosine, 5-fluorouracil, 5-bromouracil, pseudoisocytosine, 2-hydroxy-5-methyl-4-triazolopyridin, isocytosine, isoguanin, inosine and the “non-naturally occurring” nucleobases described in Benner et al., U.S. Pat. No. 5,432,272. The term “nucleobase” is intended to cover every and all of these examples as well as analogues and tautomers thereof. Especially interesting nucleobases are adenine, guanine, thymine, cytosine, and uracil, which are considered as the naturally occurring nucleobases in relation to therapeutic and diagnostic application in humans.

As used herein, “nucleoside” includes the natural nucleosides, including 2′-deoxy and 2′-hydroxyl forms, e.g., as described in Kornberg and Baker, DNA Replication, 2nd Ed. (Freeman, San Francisco, 1992).

“Analogs” in reference to nucleosides includes synthetic nucleosides having modified base moieties and/or modified sugar moieties, e.g., described generally by Scheit, Nucleotide Analogs, John Wiley, New York, 1980; Freier & Altmann, Nucl. Acid. Res., 1997, 25(22), 4429-4443, Toulmé, J. J., Nature Biotechnology 19:17-18 (2001); Manoharan M., Biochemica et Biophysica Acta 1489:117-139 (1999); Freier S., M., Nucleic Acid Research, 25:4429-4443 (1997), Uhlman, E., Drug Discovery & Development, 3: 203-213 (2000), Herdewin P., Antisense & Nucleic Acid Drug Dev., 10:297-310 (2000),); 2′-O, 3′-C-linked [3.2.0] bicycloarabinonucleosides (see e.g. N. K Christiensen., et al, J. Am. Chem. Soc., 120: 5458-5463 (1998). Such analogs include synthetic nucleosides designed to enhance binding properties, e.g., duplex or triplex stability, specificity, or the like.

As used herein, the term “analogs” when referring to amino acids refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

As used herein, the term “derivative”, when used in the context of a peptide or polypeptide, means a peptide or polypeptide different other than in primary structure (amino acids and amino acid analogs); and, when used in the context of an oligonucleotide, means an oligonucleotide different other than in the nucleotide sequence. By way of illustration, derivatives of a peptide or polypeptide may differ by being glycosylated, one form of post-translational modification. For example, peptides or polypeptides may exhibit glycosylation patterns due to expression in heterologous systems. If at least one biological activity is retained, then these peptides or polypeptides are derivatives according to the invention. Other derivatives include, but are not limited to, fusion peptides or fusion polypeptides having a covalently modified N- or C-terminus, PEGylated peptides or polypeptides, peptides or polypeptides associated with lipid moieties, alkylated peptides or polypeptides, peptides or polypeptides linked via an amino acid side-chain functional group to other peptides, polypeptides or chemicals, and additional modifications as would be understood in the art.

The term “wild-type” or “native” is used interchangeably herein and means that the nucleic acid fragment does not comprise any mutations. A “wild-type” or “native” protein means that the protein will be active at a level of activity found in nature and typically will comprise the amino acid sequence found in nature. In an aspect, the term “wild type” or “native” sequence can indicate a starting or reference sequence prior to a manipulation of the invention. A mutant or variant sequence is a sequence showing substantial variation from a wild type or reference sequence that differs from the wild type or reference sequence at one or more positions.

As used herein, the term “variant”, when used in the context of a peptide or polypeptide, means a peptide or polypeptide that differs in amino acid sequence by the insertion, deletion, or conservative substitution of amino acids, but retain at least one biological activity; and, when used in the context of an oligonucleotide, means an oligonucleotide that differs in nucleotide sequence by the insertion, deletion, or substitution of nucleotides. A particular nucleic acid sequence also implicitly encompasses “splice variants” and “allelic variants.” Similarly, a particular protein encoded by a nucleic acid implicitly encompasses any protein encoded by a splice variant or allelic variant of that nucleic acid. “Splice variants,” are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. Thus, when referring to, for example, Rad50, the term includes all variants encompassed by this definition.

As used herein, the term “homolog”, when used in the context of a peptide or polypeptide, means a peptide or polypeptide sharing a common evolutionary ancestor or having at least 50% identity thereto; and, when used in the context of an oligonucleotide, means an oligonucleotide sharing a common evolutionary ancestor or having at least 50% identity thereto.

In general, polypeptide polymorphic variants, alleles, mutants, and interspecies homologs have an amino acid sequence that has greater than about 50% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 25, 50, 100, 200, 500, 1000, or more amino acids, to a native amino acid sequence.

“Inhibitors”, “activators”, and “modulators” of Smg6, Rad50, Mre11 or Mre11/Rad50/Nbs1 polynucleotide and polypeptide sequences are used to refer to activating, inhibitory, or modulating molecules identified using in vitro and in vivo assays of each of the polynucleotide and polypeptide sequences of these molecules. Inhibitors are compounds that, e.g., bind to, partially or totally block activity, decrease, prevent, delay activation, inactivate, desensitize, or down regulate the activity or expression of Smg6, Rad50, Mre11 or Mre11/Rad50/Nbs1 proteins, e.g., antagonists. “Activators” are compounds that increase, open, activate, facilitate, enhance activation, sensitize, agonize, or up regulate Smg6, Rad50, Mre11 or Mre11/Rad50/Nbs1 protein activity, e.g., agonists. Inhibitors, activators, or modulators also include genetically modified versions of Smg6, Rad50, Mre11 or Mre11/Rad50/Nbs1 proteins, e.g., versions with altered activity, as well as naturally occurring and synthetic ligands, substrates, antagonists, agonists, antibodies, peptides, cyclic peptides, nucleic acids, antisense molecules, ribozymes, RNAi, small chemical molecules and the like.

By the term “modulate,” it is meant that any of the mentioned activities, are, e.g., increased, enhanced, increased, agonized (acts as an agonist), promoted, decreased, reduced, suppressed blocked, or antagonized (acts as an agonist). Modulation can increase activity more than 1-fold, 2-fold, 3-fold, 5-fold, 10-fold, 100-fold, etc., over baseline values. Modulation can also decrease its activity below baseline values. Modulation can also normalize an activity to a baseline value.

The phrase “stringent hybridization conditions” refers to conditions under which a polynucleotide sequence will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in lx SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

### Compositions

We have discovered that novel therapeutic target genes, Rad50, Smg6 and Mre11 play a role in cell based therapy after myocardial infarction. Specifically targeted gene therapy can increase the viability of injected cells, such as stem cells, that are used for cardiac repair. The pathways may also have direct therapeutic utility and can be manipulated by gene therapy or by a small molecule approach.

Rad50 has been described as a stem cell survival factor and as a DNA repair gene that is part of the Mre11/Rad50/Nbs1 (MRN) complex. This complex is able to generate t-loops by inserting 3′ G-overhangs at telomere ends, providing a template for telomerase. The data provided here have shown Rad50 to be overexpressed in patients, who had excellent clinical outcome after being diagnosed with new onset heart failure vs patients who did poorly. These observations lead to the hypothesis that Rad50 had regenerative capacity and was able to improve heart function over time

Stem cell based therapy after myocardial infarction is a progressive field in cardiology. Much controversy exists about the optimal type of stem cells that should be used and if additional factors, in particular cytokines, should be concurrently administered, to increase the integration and viability of the cells. Several studies reported a decrease in the number of injected stem cells in the myocardium over time. Therapeutic stimulation of Rad50, Smg6 and Mre11/Rad50/Nbs1 complex in cells via drugs or gene therapy would substantially increase the success of cell based therapy, by increasing the survival rate of administered cells.

Screening, specific selection and enrichment of these cells, with high expression of Rad50, Smg6 or Mre11 before administration via catheter would substantially increase the survival of injected cells and the success of the therapy. Furthermore, these genes would be important targets for drugs and gene therapy. Cells include stem cells, circulating blood cells, cells involved in tissue repair, remodeling and maintenance.

Screening for Rad50 and Smg6 provides clarification and optimization of cell based therapy. In addition, therapeutic targeting of those two genes would improve the response in patients. As telomerase activity is a basic process, those genes could be targeted in cell based therapies in general.

In a preferred embodiment, Rad50, Smg6, Mre11, Nbs1, or the Mre11/Rad50/Nbs1 complex, expression is targeted by pharmacologic agents or genetic agents which modulate the expression of these molecules in cells. These cells include, for example, stem cells, circulating blood cells, myocytes, or any type of cell involved in repair and maintenance mechanisms, which can be transformed with vectors expressing Rad50, Smg6, Mre11, Nbs1, or the Mre11/Rad50/Nbs1 complex, variants, mutants, and fragments thereof. For example, a method of producing a cell expressing Smg6 and/or Rad50, variants, mutants, and fragments thereof, comprises a DNA construct comprising the above DNA sequences; a DNA sequence encoding at least one selectable marker; administering to the cell the DNA construct; maintaining the cell under conditions appropriate for maintaining the construct, and, culturing the recombinant cell under conditions appropriate for propagating the recombinant cell.

In another preferred embodiment, a method of identifying candidate agents comprises culturing a cell in proliferating medium; exposing the cells to the candidate agent; and observing the effect of the candidate agent on the expression of Rad50, Smg6, Mre11, Nbs1, and/or the Mre11/Rad50/Nbs1 complex, enzymes or factors involved in the generation oft-loops variants, mutants, and fragments thereof. The effects of the candidate agent include, but not limited to changes in nucleic acid expression, protein expression, nucleic acid, polypeptide trafficking, t-loop generation, stem cell trafficking and the like.

In another preferred embodiment, cells are modified to overexpress Smg6 and/or Rad50 and/or Mre11 and/or the Mre11/Rad50/Nbs1 complex, enzymes or factors involved in the generation of t-loops. These molecules represent novel therapeutic targets which can be manipulated by gene therapy or small molecule strategies. Furthermore, since these pathways are important in stem cell viability, modifying stem cells to overexpress these pathways would enhance stem cell effectiveness for tissue repair. In other preferred embodiments, the cell can be any type of cell, for example, circulating blood cells such as monocytes, lymphocytes; cells involved in repair and remodeling mechanisms.

In a preferred embodiment, a composition of biomarkers comprises Smg6, Rad50 and Mre11. The biomarker composition can include at least one marker comprising a polynucleotide, fragments, alleles, variants, mutants, substituted polynucleotides and complementary sequences thereof of the Smg6 and/or Rad50 and/or Mre11. In some aspects, the Smg6, Rad50 and Mre11 polynucleotides comprise one or more substituted nucleic acid bases.

The biomarker composition can also include at least one marker comprising a polypeptide, fragment, variants, analogs and mutants thereof of Rad50, Smg6, Mre11, Nbs1, or the Mre11/Rad50/Nbs1 complex. In some aspects, the polypeptides comprise one or more non natural amino acids.

A “non-natural amino acid” refers to an amino acid that is not one of the 20 common amino acids or pyrolysine or selenocysteine. Other terms that may be used synonymously with the term “non-natural amino acid” is “non-naturally encoded amino acid,” “unnatural amino acid,” “non-naturally-occurring amino acid,” and variously hyphenated and non-hyphenated versions thereof. The term “non-natural amino acid” includes, but is not limited to, amino acids which occur naturally by modification of a naturally encoded amino acid (including but not limited to, the 20 common amino acids or pyrrolysine and selenocysteine) but are not themselves incorporated, without user manipulation, into a growing polypeptide chain by the translation complex. Examples of naturally-occurring amino acids that are not naturally-encoded include, but are not limited to, N-acetylglucosaminyl-L-serine, N-acetylglucosaminyl-L-threonine, and O-phosphotyrosine. Additionally, the term “non-natural amino acid” includes, but is not limited to, amino acids which do not occur naturally and may be obtained synthetically or may be obtained by modification of non-natural amino acids.

In some cases, the non-natural amino acid substitution(s) or incorporation(s) will be combined with other additions, substitutions, or deletions within the polypeptide to affect other chemical, physical, pharmacologic and/or biological traits. In some cases, the other additions, substitutions or deletions may increase the stability (including but not limited to, resistance to proteolytic degradation) of the polypeptide or increase affinity of the polypeptide for its appropriate receptor, ligand and/or binding proteins. In some cases, the other additions, substitutions or deletions may increase the solubility of the polypeptide. In some embodiments sites are selected for substitution with a naturally encoded or non-natural amino acid in addition to another site for incorporation of a non-natural amino acid for the purpose of increasing the polypeptide solubility following expression in recombinant host cells. In some embodiments, the polypeptides comprise another addition, substitution, or deletion that modulates affinity for the associated ligand, binding proteins, and/or receptor, modulates (including but not limited to, increases or decreases) receptor dimerization, stabilizes receptor dimers, modulates circulating half-life, modulates release or bio-availability, facilitates purification, or improves or alters a particular route of administration. Similarly, the non-natural amino acid polypeptide can comprise chemical or enzyme cleavage sequences, protease cleavage sequences, reactive groups, antibody-binding domains (including but not limited to, FLAG or poly-His) or other affinity based sequences (including but not limited to, FLAG, poly-His, GST, etc.) or linked molecules (including but not limited to, biotin) that improve detection (including but not limited to, GFP), purification, transport thru tissues or cell membranes, prodrug release or activation, size reduction, or other traits of the polypeptide.

The methods and compositions described herein include incorporation of one or more non-natural amino acids into a polypeptide. One or more non-natural amino acids may be incorporated at one or more particular positions which does not disrupt activity of the polypeptide. This can be achieved by making “conservative” substitutions, including but not limited to, substituting hydrophobic amino acids with non-natural or natural hydrophobic amino acids, bulky amino acids with non-natural or natural bulky amino acids, hydrophilic amino acids with non-natural or natural hydrophilic amino acids) and/or inserting the non-natural amino acid in a location that is not required for activity.

A variety of biochemical and structural approaches can be employed to select the desired sites for substitution with a non-natural amino acid within the polypeptide. Any position of the polypeptide chain is suitable for selection to incorporate a non-natural amino acid, and selection may be based on rational design or by random selection for any or no particular desired purpose. Selection of desired sites may be based on producing a non-natural amino acid polypeptide (which may be further modified or remain unmodified) having any desired property or activity, including but not limited to agonists, super-agonists, partial agonists, inverse agonists, antagonists, receptor binding modulators, receptor activity modulators, modulators of binding to binder partners, binding partner activity modulators, binding partner conformation modulators, dimer or multimer formation, no change to activity or property compared to the native molecule, or manipulating any physical or chemical property of the polypeptide such as solubility, aggregation, or stability. For example, locations in the polypeptide required for biological activity of a polypeptide can be identified using methods including, but not limited to, point mutation analysis, alanine scanning or homolog scanning methods. Residues other than those identified as critical to biological activity by methods including, but not limited to, alanine or homolog scanning mutagenesis, may be good candidates for substitution with a non-natural amino acid depending on the desired activity sought for the polypeptide. Alternatively, the sites identified as being involved in a biological activity may also be good candidates for substitution with a non-natural amino acid, again depending on the desired activity sought for the polypeptide. Another alternative would be to make serial substitutions in each position on the polypeptide chain with a non-natural amino acid and observe the effect on the activities of the polypeptide. Any means, technique, or method for selecting a position for substitution with a non-natural amino acid into any polypeptide is suitable for use in the methods, techniques and compositions described herein.

The structure and activity of naturally-occurring mutants of a polypeptide that contain deletions can also be examined to determine regions of the protein that are likely to be tolerant of substitution with a non-natural amino acid. Once residues that are likely to be intolerant to substitution with non-natural amino acids have been eliminated, the impact of proposed substitutions at each of the remaining positions can be examined using methods including, but not limited to, the three-dimensional structure of the relevant polypeptide, and any associated ligands or binding proteins. X-ray crystallographic and NMR structures of many polypeptides are available in the Protein Data Bank (PDB, www.rcsb.org), a centralized database containing three-dimensional structural data of large molecules of proteins and nucleic acids, one can be used to identify amino acid positions that can be substituted with non-natural amino acids. In addition, models may be made investigating the secondary and tertiary structure of polypeptides, if three-dimensional structural data is not available. Thus, the identity of amino acid positions that can be substituted with non-natural amino acids can be readily obtained. Exemplary sites of incorporation of a non-natural amino acid include, but are not limited to, those that are excluded from potential receptor binding regions, or regions for binding to binding proteins or ligands may be fully or partially solvent exposed, have minimal or no hydrogen-bonding interactions with nearby residues, may be minimally exposed to nearby reactive residues, and/or may be in regions that are highly flexible as predicted by the three-dimensional crystal structure of a particular polypeptide with its associated receptor, ligand or binding proteins.

A wide variety of non-natural amino acids can be substituted for, or incorporated into, a given position in a polypeptide. By way of example, a particular non-natural amino acid may be selected for incorporation based on an examination of the three dimensional crystal structure of a polypeptide with its associated ligand, receptor and/or binding proteins, a preference for conservative substitutions.

Antibodies and Aptamers: Antibodies that specifically bind to anyone of Rad50, Smg6, Mre11, Nbs1, or the Mre11/Rad50/Nbs1 complex, fragments, mutants, variants and complementary sequences thereof are provided. Such antibodies may be used in methods of isolating pure, for example, Rad50, and in methods of differentiating between individuals with a good prognosis vs. a bad prognosis for recovery and long term survival from heart failure and other cardiac disorders. That is, by identifying individuals whose tissue shows an absence or deficiency in, for example Rad50, as compared to a normal subject, a bad prognosis for survival is indicated.

As used herein, the term “aptamer” or “selected nucleic acid binding species” include non-modified or chemically modified RNA or DNA. The method of selection may be by, but is not limited to, affinity chromatography and the method of amplification by reverse transcription (RT) or polymerase chain reaction (PCR).

As used herein, the term “antibody” is meant to refer to complete, intact antibodies, and Fab fragments and F(ab)₂ fragments thereof. Complete, intact antibodies include monoclonal antibodies such as murine monoclonal antibodies, chimeric antibodies and humanized antibodies. Antibodies that bind to an epitope which is present on, for example, Rad50, are useful to isolate and purify the Rad50 from both natural sources and recombinant expression systems using well known techniques such as affinity chromatography. Such antibodies are useful to detect the presence of such protein in a sample and to determine if cells are expressing the protein.

The production of antibodies and the protein structures of complete, intact antibodies, Fab fragments and F(ab)₂ fragments and the organization of the genetic sequences that encode such molecules are well known and are described, for example, in Harlow, E. and D. Lane (1988) Antibodies: A Laboratory Manual, Cold Spring Harbor Laboratory, Cold Spring Harbor, N.Y., which is incorporated herein by reference. Briefly, for example, Rad50 or an immunogenic fragment thereof, is injected into mice. The spleen of the mouse is removed; the spleen cells are isolated and fused with immortalized mouse cells. The hybrid cells, or hybridomas, are cultured and those cells which secrete antibodies are selected. The antibodies are analyzed and, if found to specifically bind to protein, the hybridoma which produces them is cultured to produce a continuous supply of antibodies.

### Cellular Compositions

Stem Cells: This invention can be practiced using stem cells of various types. Except where otherwise required, the invention can be practiced using stem cells of any vertebrate species. Included are stem cells from humans; as well as non-human primates, domestic animals, livestock, and other non-human mammals.

In a preferred embodiment, progenitor cells are isolated from a patient. However, the stem cells can be donor derived. Amongst the stem cells suitable for use in this invention are primate pluripotent stem (pPS) cells derived from tissue formed after gestation, such as a blastocyst, or fetal or embryonic tissue taken any time during gestation. Non-limiting examples are primary cultures or established lines of embryonic stem cells or embryonic germ cells.

Embryonic Stem Cells: Embryonic stem cells can be isolated from blastocysts of members of the primate species (Thomson et al., Proc. Natl. Acad. Sci. USA 92:7844, 1995). Human embryonic stem (hES) cells can be prepared from human blastocyst cells using the techniques described by Thomson et al. (U.S. Pat. No. 5,843,780; Science 282:1145, 1998; Curr. Top. Dev. Biol. 38:133 ff., 1998) and Reubinoff et al, Nature Biotech. 18:399 (2000)).

Briefly, human blastocysts are obtained from human in vivo preimplantation embryos. Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses are isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers.

After 9 to 15 days, inner cell mass-derived outgrowths are dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology are individually selected by micropipette, mechanically dissociated into clumps, and replated. ES-like morphology is characterized as compact colonies with apparently high nucleus to cytoplasm ratio and prominent nucleoli. Resulting ES cells are then routinely split every 1-2 weeks by brief trypsinization, exposure to Dulbecco's PBS (containing 2 mM EDTA), exposure to type IV collagenase (about 200 U/mL; Gibco) or by selection of individual colonies by micropipette. Clump sizes of about 50 to 100 cells are optimal.

In a preferred embodiment, isolated stem cells are contacted with Rad50, Smg6, Mre11, Nbs1, or the Mre11/Rad50/Nbs1 complex, enzymes or factors involved in the generation of t-loops. The molecules can be introduced into the stem cells by, for example, expression vectors encoding Rad50, Smg6, Mre11, Nbs1, and/or the Mre11/Rad50/Nbs1 complex.

In another preferred embodiment, the stem cells are treated with an agent that modulates the expression of Rad50, Smg6, Mre11, Nbs1, or the Mre11/Rad50/Nbs1 complex.

As used herein, “expression” includes the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989). Similarly, a eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Vectors include plasmids, phages, viruses, etc.

Retroviral vectors typically comprise the RNA of a transmissible agent, into which a heterologous sequence encoding a protein of interest is inserted. Typically, the retroviral RNA genome is expressed from a DNA constrict. A common way to insert one segment of DNA into another segment of DNA involves the use of enzymes called restriction enzymes that cleave DNA at specific sites (specific groups of nucleotides) called restriction sites. A “cassette” refers to a DNA segment that can be inserted into a vector or into another piece of DNA at a defined restriction site. Preferably, a cassette is an “expression cassette” in which the DNA is a coding sequence or segment of DNA that codes for an expression product that can be inserted into a vector at defined restriction sites. The cassette restriction sites generally are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of DNA construct is a “plasmid” that generally is a self-contained molecule of double-stranded DNA, usually of bacterial origin, that can readily accept additional (foreign) DNA and which can be readily introduced into a suitable producer cell. A plasmid vector often contains coding DNA and promoter DNA and has one or more restriction sites suitable for inserting foreign DNA. A large number of vectors, including plasmid and fungal vectors, have been described for replication and/or expression in a variety of eukaryotic and prokaryotic hosts. Non-limiting examples include pKK plasmids (Amersham Pharmacia Biotech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes. A “retroviral plasmid vector” means a plasmid which includes all or part of a retroviral genome including 5′ and 3′ retroviral long-term repeat (LTR) sequences, a packaging signal (ψ), and may include one or more polynucleotides encoding a protein(s) or polypeptide(s) of interest, such as a therapeutic agent or a selectable marker. Such retroviral plasmid vectors are described, e.g., in U.S. Pat. No. 5,952,225 which is specifically incorporated herein by reference.

Expression of a SMG6, RAD50, SMG6 and RAD50, and/or the Mre11/Rad50/Nbs1 complex protein, fragment thereof, conservative variant thereof, or analog or derivative thereof of the invention may be controlled by promoter/enhancer elements disclosed herein, but these regulatory elements must be functional in the host selected for expression. Promoters which may be used to control gene expression include but are not limited to, the SV40 early promoter region (Benoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42); prokaryotic expression vectors such as the β-lactamase promoter (Villa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25); promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity.

Mammalian expression vectors contemplated for use in the invention include vectors with inducible promoters, such as the dihydrofolate reductase (DHFR) promoter, e.g., any expression vector with a DHFR expression vector, or a DHFR/methotrexate co-amplification vector, such as pED (PstI, SalI, SbaI, SmaI, and EcoRI cloning site, with the vector expressing both the cloned gene and DHFR, see Kaufman, Current Protocols in Molecular Biology, 16.12 (1991). Alternatively, a glutamine synthetase/methionine sulfoximine co-amplification vector, such as pEE14 (HindIII, XbaI, SmaI, SbaI, EcoRI, and BclI cloning site, in which the vector expresses glutamine synthase and the cloned gene; Celltech). In another embodiment, a vector that directs episomal expression under control of Epstein Barr Virus (EBV) can be used, such as pREP4 (BamHI, SfiI, XhoI, NotI, NheI, HindIII, NheI, PvuII, and KpnI cloning site, constitutive RSV-LTR promoter, hygromycin selectable marker; Invitrogen), pCEP4 (BamHI, SfiI, XhoI, NotI, NheI, HindIII, NheI, PvuII, and KpnI cloning site, constitutive hCMV immediate early gene, hygromycin selectable marker; Invitrogen), pMEP4 (KpnI, PvuI, NheI, HindIII, NotI, XhoI, SfiI, BamHII cloning site, inducible metallothionein IIa gene promoter, hygromycin selectable marker; Invitrogen), pREP8 (BamHI, XhoI, NotI, HindIII, NheI, and KpnI cloning site, RSV-LTR promoter, histidinol selectable marker; Invitrogen), pREP9 (KpnI, NheI, HindIII, NotI, XhoI, SfiI, and BamHI cloning site, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen). Selectable mammalian expression vectors for use in the invention include pRc/CMV (HindIII, BstXI, NotI, SbaI, and ApaI cloning site, G418 selection; Invitrogen), pRc/RSV (HindIII, SpeI, BstXI, NotI, XbaI cloning site, G418 selection; Invitrogen), and others. Vaccinia virus mammalian expression vectors (see, Kaufman, 1991, supra) for use according to the invention include but are not limited to pSCII (SmaI cloning site, TK- and β-gal selection), pMJ601 (SalI, SmaI, Nil, NarI, BspMII, BamHI, ApaI, NheI, SacII, KpnI, and HindIII cloning site; TK- and β-gal selection), and pTKgptFIS (EcoRI, PsiI, SalI, AccI, HindIII, SbaI, BamHI, and Hpa cloning site, TK or XPRT selection).

The molecules of interest, e.g. SMG6, RAD50, Mre11, Mre11/Rad50/Nbs1 are operably linked to the promoter. A nucleic acid is “operably linked” when it is placed into a functional relationship with another nucleic acid sequence. For instance, a promoter or enhancer is operably linked to a coding sequence if it increases the transcription of the coding sequence. Operably linked means that the DNA sequences being linked are typically contiguous and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

Vectors are introduced into desired host cells by methods known in the art, e.g., transfection, electroporation, microinjection, transduction, cell fusion, DEAE dextran, calcium phosphate precipitation, lipofection (lysosome fusion), use of a gene gun, or a DNA vector transporter.

Synthetic vector particles can be prepared using lipofection technology, optionally with other transfection facilitating agents (peptides, polymers, etc.). Synthetic cationic lipids can be used to prepare liposomes for in vivo transfection of a gene (Felgner, et. al., Proc. Natl. Acad. Sci. USA 1987, 84:7413-7417; Felgner and Ringold, Science 1989, 337:387-388; see Mackey, et al., Proc. Natl. Acad. Sci. USA 1988, 85:8027-8031; Ulmer et al., Science 1993, 259:1745-1748). Useful lipid compounds and compositions for transfer of nucleic acids are described in PCT Publications WO95/18863 and WO96/17823, and in U.S. Pat. No. 5,459,127. Lipids may be chemically coupled to molecules for the purpose of targeting (see Mackey, et al., supra), or by insertion of a polypeptide construct into the lipid bilayer, i.e., by analogy to a transmembrane protein.

Circulating Blood Cells and Cells Involved in Tissue Repair, Remodeling and Maintenance: The data provided herein, show the expression of Rad50 in circulation. Briefly, we have shown that Rad50 expression levels are strongly involved in response to ischemic injury both in heart tissue as well as circulating blood cells. These observations strongly support our hypothesis that RAD50 plays an important regenerative role in patients with heart failure and may be a promising therapeutic target, to support DNA repair, preserve genome stability and enhance regenerative capacity of stem cells and other types of cells that are involved in remodeling and repair mechanisms.

In preferred embodiments, detection in circulating cells of a biomarker composition comprising markers: Rad50, Smg6, Mre11, Nbs1, or the Mre11/Rad50/Nbs1 complex, are prognostic of the survival outcome in patients who have had heart failure.

In preferred embodiments, detection in cells of a biomarker composition comprising markers: Rad50, Smg6, Mre11, Nbs1, or the Mre11/Rad50/Nbs1 complex, are prognostic of the survival outcome in patients who have had heart failure.

Circulating cells include cells of the immune system such as lymphocytes, monocytes, mast cells, granulocytes and the like. Cells involved in tissue repair, remodeling or maintenance, include, for example, mast cells, fibroblasts, heart valve interstitial cells (VICs), stem cells, and the like. These cells are involved in often complex repair mechanisms which can include recruitment of cells, secretion of factors such as for example metalloproteinases, cytokines, growth factors, angiotensins, etc; or cell surface expression of receptors, ligands and the like. Expression profiling data show a rich collection of proteins from a variety of distinct pathways (i.e., cytokines, chemokines, angiogenic factors, and an extensive presence of protease inhibitors that moderate tissue remodeling) are secreted by a variety of cells. For example, wound healing and normal tissue remodeling are believed to be affected by the level of metalloproteinase activity available at the relevant site and the regulation thereof. Woessner, J. F. Biochemical Journal, 161, 535-542 (1977); Woessner, J. F. FASEB Journal, 5, 2145-2154 (1991); Herron, et al. J. Biol. Chem., 261, 2810-2813 (1986). Healing of wounds, for example, involves substantial remodeling of tissue. Although such a process is not invasive per se, it involves localized breakdown of extracellular matrix and the breaking and forming of cell attachments.

Other types of cells include, for example, skeletal myoblasts which are cells responsible for the regeneration of skeletal muscle following injury. These cells have the ability to fuse with other myoblasts or damaged muscle fibers. These cells can be treated with an agent that induces expression of Rad50, Smg6, Mre11, Nbs1, or the Mre11/Rad50/Nbs1 complex, or transformed with a vector that expresses Rad50, Smg6, Mre11, Nbs1, or the Mre11/Rad50/Nbs1 complex, and administered to a patient's damaged myocardium or other damaged muscle tissue, and improve tissue properties or functionally participate in contraction. Go cells are also isolated from adult skeletal muscle, and these non-satellite cells express GATA-4 and, under in vitro growth conditions, develop into spontaneously beating cardiomyocyte-like cells.

### Candidate Therapeutic Agents

In a preferred embodiment Rad50, Smg6, Mre11, Nbs1, or the Mre11/Rad50/Nbs1 complex, enzymes or factors involved in the generation of t-loops, expression is modulated in a cell such as stem cells, by a candidate therapeutic agent.

In one embodiment, screening comprises contacting a cell culture, for example, which can comprise a vector expressing Rad50, Smg6, Mre11, Nbs1, or the Mre11/Rad50/Nbs1 complex, enzymes or factors involved in the generation of t-loops, with a diverse library of member compounds. The compounds or “candidate therapeutic agents” can be any organic, inorganic, small molecule, protein, antibody, aptamer, nucleic acid molecule, or synthetic compound.

Candidate agents include numerous chemical classes, though typically they are organic compounds including small organic compounds, nucleic acids including oligonucleotides, and peptides. Small organic compounds suitably may have e.g. a molecular weight of more than about 40 or 50 yet less than about 2,500. Candidate agents may comprise functional chemical groups that interact with proteins and/or DNA.

Candidate agents may be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of e.g. bacterial, fungal and animal extracts are available or readily produced.

Chemical Libraries: Developments in combinatorial chemistry allow the rapid and economical synthesis of hundreds to thousands of discrete compounds. These compounds are typically arrayed in moderate-sized libraries of small molecules designed for efficient screening. Combinatorial methods, can be used to generate unbiased libraries suitable for the identification of novel compounds. In addition, smaller, less diverse libraries can be generated that are descended from a single parent compound with a previously determined biological activity. In either case, the lack of efficient screening systems to specifically target therapeutically relevant biological molecules produced by combinational chemistry such as inhibitors of important enzymes hampers the optimal use of these resources.

A combinatorial chemical library is a collection of diverse chemical compounds generated by either chemical synthesis or biological synthesis, by combining a number of chemical “building blocks,” such as reagents. For example, a linear combinatorial chemical library, such as a polypeptide library, is formed by combining a set of chemical building blocks (amino acids) in a large number of combinations, and potentially in every possible way, for a given compound length (i.e., the number of amino acids in a polypeptide compound). Millions of chemical compounds can be synthesized through such combinatorial mixing of chemical building blocks.

A “library” may comprise from 2 to 50,000,000 diverse member compounds. Preferably, a library comprises at least 48 diverse compounds, preferably 96 or more diverse compounds, more preferably 384 or more diverse compounds, more preferably, 10,000 or more diverse compounds, preferably more than 100,000 diverse members and most preferably more than 1,000,000 diverse member compounds. By “diverse” it is meant that greater than 50% of the compounds in a library have chemical structures that are not identical to any other member of the library. Preferably, greater than 75% of the compounds in a library have chemical structures that are not identical to any other member of the collection, more preferably greater than 90% and most preferably greater than about 99%.

The preparation of combinatorial chemical libraries is well known to those of skill in the art. For reviews, see Thompson et al., Synthesis and application of small molecule libraries, Chem Rev 96:555-600, 1996; Kenan et al., Exploring molecular diversity with combinatorial shape libraries, Trends Biochem Sci 19:57-64, 1994; Janda, Tagged versus untagged libraries: methods for the generation and screening of combinatorial chemical libraries, Proc Natl Acad Sci USA. 91:10779-85, 1994; Lebl et al., One-bead-one-structure combinatorial libraries, Biopolymers 37:177-98, 1995; Eichler et al., Peptide, peptidomimetic, and organic synthetic combinatorial libraries, Med Res Rev. 15:481-96, 1995; Chabala, Solid-phase combinatorial chemistry and novel tagging methods for identifying leads, Curr Opin Biotechnol. 6:632-9, 1995; Dolle, Discovery of enzyme inhibitors through combinatorial chemistry, Mol Divers. 2:223-36, 1997; Fauchere et al., Peptide and nonpeptide lead discovery using robotically synthesized soluble libraries, Can J. Physiol Pharmacol. 75:683-9, 1997; Eichler et al., Generation and utilization of synthetic combinatorial libraries, Mol Med Today 1: 174-80, 1995; and Kay et al., Identification of enzyme inhibitors from phage-displayed combinatorial peptide libraries, Comb Chem High Throughput Screen 4:535-43, 2001.

Other chemistries for generating chemical diversity libraries can also be used. Such chemistries include, but are not limited to, peptoids (PCT Publication No. WO 91/19735); encoded peptides (PCT Publication WO 93/20242); random bio-oligomers (PCT Publication No. WO 92/00091); benzodiazepines (U.S. Pat. No. 5,288,514); diversomers, such as hydantoins, benzodiazepines and dipeptides (Hobbs, et al., Proc. Nat. Acad. Sci. USA, 90:6909-6913 (1993)); vinylogous polypeptides (Hagihara, et al., J. Amer. Chem. Soc. 114:6568 (1992)); nonpeptidal peptidomimetics with β-D-glucose scaffolding (Hirschmann, et al., J. Amer. Chem. Soc., 114:9217-9218 (1992)); analogous organic syntheses of small compound libraries (Chen, et al., J. Amer. Chem. Soc., 116:2661 (1994)); oligocarbamates (Cho, et al., Science, 261:1303 (1993)); and/or peptidyl phosphonates (Campbell, et al., J. Org. Chem. 59:658 (1994)); nucleic acid libraries (see, Ausubel, Berger and Sambrook, all supra); peptide nucleic acid libraries (see, e.g., U.S. Pat. No. 5,539,083); antibody libraries (see, e.g., Vaughn, et al., Nature Biotechnology, 14(3):309-314 (1996) and PCT/US96/10287); carbohydrate libraries (see, e.g., Liang, et al., Science, 274:1520-1522 (1996) and U.S. Pat. No. 5,593,853); small organic molecule libraries (see, e.g., benzodiazepines, Baum C&E News, January 18, page 33 (1993); isoprenoids (U.S. Pat. No. 5,569,588); thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337); benzodiazepines (U.S. Pat. No. 5,288,514); and the like.

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd., Moscow, RU, 3D Pharmaceuticals, Exton, Pa., Martek Bio sciences, Columbia, Md., etc.).

Small Molecules: Small molecule test compounds can initially be members of an organic or inorganic chemical library. As used herein, “small molecules” refers to small organic or inorganic molecules of molecular weight below about 3,000 Daltons. The small molecules can be natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio., 1:60 (1997). In addition, a number of small molecule libraries are commercially available.

In a preferred embodiment, the compounds are assayed against the cells comprising Smg6 and/or Rad50, the Mre11/Rad50/Nbs1 complex, enzymes or factors involved in the generation of t-loops as high throughput screening. The reporter molecules can be the same or different molecules, however, the reporter molecules are preferably different.

In another aspect, the present invention provides a method for analyzing cells comprising providing an array of locations which contain multiple cells wherein the cells contain one or more fluorescent or luciferase reporter molecules; scanning multiple cells in each of the locations containing cells to obtain signals from the reporter molecule in the cells; converting the signals into digital data; and utilizing the digital data to determine the distribution, environment or activity of the reporter molecule within the cells.

A major component of the new drug discovery paradigm is a continually growing family of fluorescent and luminescent reagents that are used to measure the temporal and spatial distribution, content, and activity of intracellular ions, metabolites, macromolecules, and organelles. Classes of these reagents include labeling reagents that measure the distribution and amount of molecules in living and fixed cells, environmental indicators to report signal transduction events in time and space, and fluorescent protein biosensors to measure target molecular activities within living cells. A multiparameter approach that combines several reagents in a single cell is a powerful new tool for drug discovery.

This method relies on the high affinity of fluorescent or luminescent molecules for specific cellular components. The affinity for specific components is governed by physical forces such as ionic interactions, covalent bonding (which includes chimeric fusion with protein-based chromophores, fluorophores, and lumiphores), as well as hydrophobic interactions, electrical potential, and, in some cases, simple entrapment within a cellular component. The luminescent probes can be small molecules, labeled macromolecules, or genetically engineered proteins, including, but not limited to green fluorescent protein chimeras.

Those skilled in this art will recognize a wide variety of fluorescent reporter molecules that can be used in the present invention, including, but not limited to, fluorescently labeled biomolecules such as proteins, phospholipids, RNA and DNA hybridizing probes. Similarly, fluorescent reagents specifically synthesized with particular chemical properties of binding or association have been used as fluorescent reporter molecules (Barak et al., (1997), J. Biol. Chem. 272:27497-27500; Southwick et al., (1990), Cytometry 11:418-430; Tsien (1989) in Methods in Cell Biology, Vol. 29 Taylor and Wang (eds.), pp. 127-156). Fluorescently labeled antibodies are particularly useful reporter molecules due to their high degree of specificity for attaching to a single molecular target in a mixture of molecules as complex as a cell or tissue.

The luminescent probes can be synthesized within the living cell or can be transported into the cell via several non-mechanical modes including diffusion, facilitated or active transport, signal-sequence-mediated transport, and endocytotic or pinocytotic uptake. Mechanical bulk loading methods, which are well known in the art, can also be used to load luminescent probes into living cells (Barber et al. (1996), Neuroscience Letters 207:17-20; Bright et al. (1996), Cytometry 24:226-233; McNeil (1989) in Methods in Cell Biology, Vol. 29, Taylor and Wang (eds.), pp. 153-173). These methods include electroporation and other mechanical methods such as scrape-loading, bead-loading, impact-loading, syringe-loading, hypertonic and hypotonic loading. Additionally, cells can be genetically engineered to express reporter molecules, such as GFP, coupled to a protein of interest as previously described (Chalfie and Prasher U.S. Pat. No. 5,491,084; Cubitt et al. (1995), Trends in Biochemical Science 20:448-455).

Once in the cell, the luminescent probes accumulate at their target domain as a result of specific and high affinity interactions with the target domain or other modes of molecular targeting such as signal-sequence-mediated transport. Fluorescently labeled reporter molecules are useful for determining the location, amount and chemical environment of the reporter. For example, whether the reporter is in a lipophilic membrane environment or in a more aqueous environment can be determined (Giuliano et al. (1995), Ann. Rev. of Biophysics and Biomolecular Structure 24:405-434; Giuliano and Taylor (1995), Methods in Neuroscience 27.1-16). The pH environment of the reporter can be determined (Bright et al. (1989), J. Cell Biology 104:1019-1033; Giuliano et al. (1987), Anal. Biochem. 167:362-371; Thomas et al. (1979), Biochemistry 18:2210-2218). It can be determined whether a reporter having a chelating group is bound to an ion, such as Ca⁺⁺, or not (Bright et al. (1989), In Methods in Cell Biology, Vol. 30, Taylor and Wang (eds.), pp. 157-192; Shimoura et al. (1988), J. of Biochemistry (Tokyo) 251:405-410; Tsien (1989) In Methods in Cell Biology, Vol. 30, Taylor and Wang (eds.), pp. 127-156).

Furthermore, certain cell types within an organism may contain components that can be specifically labeled that may not occur in other cell types. Therefore, reporter molecules can be designed to label not only specific components within specific cells, but also specific cells within a population of mixed cell types.

Those skilled in the art will recognize a wide variety of ways to measure fluorescence. For example, some fluorescent reporter molecules exhibit a change in excitation or emission spectra, some exhibit resonance energy transfer where one fluorescent reporter loses fluorescence, while a second gains in fluorescence, some exhibit a loss (quenching) or appearance of fluorescence, while some report rotational movements (Giuliano et al. (1995), Ann. Rev. of Biophysics and Biomol. Structure 24:405-434; Giuliano et al. (1995), Methods in Neuroscience 27:1-16).

The whole procedure can be fully automated. For example, sampling of sample materials may be accomplished with a plurality of steps, which include withdrawing a sample from a sample container and delivering at least a portion of the withdrawn sample to test cell culture (e.g., a cell culture wherein gene expression is regulated). Sampling may also include additional steps, particularly and preferably, sample preparation steps. In one approach, only one sample is withdrawn into the auto-sampler probe at a time and only one sample resides in the probe at one time. In other embodiments, multiple samples may be drawn into the auto-sampler probe separated by solvents. In still other embodiments, multiple probes may be used in parallel for auto sampling.

In the general case, sampling can be effected manually, in a semi-automatic manner or in an automatic manner. A sample can be withdrawn from a sample container manually, for example, with a pipette or with a syringe-type manual probe, and then manually delivered to a loading port or an injection port of a characterization system. In a semi-automatic protocol, some aspect of the protocol is effected automatically (e.g., delivery), but some other aspect requires manual intervention (e.g., withdrawal of samples from a process control line). Preferably, however, the sample(s) are withdrawn from a sample container and delivered to the characterization system, in a fully automated manner—for example, with an auto-sampler.

In one embodiment, auto-sampling may be done using a microprocessor controlling an automated system (e.g., a robot arm). Preferably, the microprocessor is user-programmable to accommodate libraries of samples having varying arrangements of samples (e.g., square arrays with “n-rows” by “n-columns,” rectangular arrays with “n-rows” by “m-columns,” round arrays, triangular arrays with “r-” by “r-” by “r-” equilateral sides, triangular arrays with “r-base” by “s-” by “s-” isosceles sides, etc., where n, m, r, and s are integers).

Automated sampling of sample materials optionally may be effected with an auto-sampler having a heated injection probe (tip). An example of one such auto sampler is disclosed in U.S. Pat. No. 6,175,409 B1 (incorporated by reference).

According to the present invention, one or more systems, methods or both are used to identify a plurality of sample materials. Though manual or semi-automated systems and methods are possible, preferably an automated system or method is employed. A variety of robotic or automatic systems are available for automatically or programmably providing predetermined motions for handling, contacting, dispensing, or otherwise manipulating materials in solid, fluid liquid or gas form according to a predetermined protocol. Such systems may be adapted or augmented to include a variety of hardware, software or both to assist the systems in determining mechanical properties of materials. Hardware and software for augmenting the robotic systems may include, but are not limited to, sensors, transducers, data acquisition and manipulation hardware, data acquisition and manipulation software and the like. Exemplary robotic systems are commercially available from CAVRO Scientific Instruments (e.g., Model NO. RSP9652) or BioDot (Microdrop Model 3000).

Generally, the automated system includes a suitable protocol design and execution software that can be programmed with information such as synthesis, composition, location information or other information related to a library of materials positioned with respect to a substrate. The protocol design and execution software is typically in communication with robot control software for controlling a robot or other automated apparatus or system. The protocol design and execution software is also in communication with data acquisition hardware/software for collecting data from response measuring hardware. Once the data is collected in the database, analytical software may be used to analyze the data, and more specifically, to determine properties of the candidate drugs, or the data may be analyzed manually.

### Diagnostics and Kits

In a preferred embodiment, the invention provides for prognosis and diagnosis of heart failure and other cardiac disorders. In another preferred embodiment, a biomarker composition for the diagnosis of heart failure and cardiac diseases comprising markers Rad50, Smg6, and Mre11 and combinations thereof. In one aspect, the biomarker further comprises a marker selected from the group consisting of Mre11, Nbs1, Mre11/Rad50/Nbs1 complex, enzymes and t-loop generation factors.

In another preferred embodiment, the biomarker composition for the diagnosis of heart failure and cardiac diseases comprises the Rad50, Smg6, and Mre11 markers and at least one marker comprising a polynucleotide, fragments, alleles, variants, mutants, complementary sequences thereof of Rad50, Smg6, and Mre11.

In another preferred embodiment, the biomarker composition for the diagnosis of heart failure and cardiac diseases further comprises the Rad50, Smg6, and Mre11 markers and at least one marker comprising a polypeptide, fragment, variants, analogs and mutants thereof of Rad50, Smg6, and Mre11.

In another preferred embodiment, the biomarker composition for the diagnosis of heart failure and cardiac diseases further comprises the Rad50, Smg6, and Mre11 markers and at least one marker comprising a polynucleotide, fragments, alleles, variants, mutants substituted polynucleotides and complementary sequences thereof of Smg6.

In another preferred embodiment, the biomarker composition for the diagnosis of heart failure and cardiac diseases further comprises the Rad50, Smg6, and Mre11 markers and at least one marker comprising a polypeptide, fragment, variants, analogs and mutants thereof of Smg6.

In another preferred embodiment, the biomarker composition for the diagnosis of heart failure and cardiac diseases further comprises the Rad50, Smg6, and Mre11 markers and at least one marker comprising a polynucleotide, fragments, alleles, variants, mutants, substituted polynucleotides and complementary sequences thereof of Rad50.

In another preferred embodiment, the biomarker composition for the diagnosis of heart failure and cardiac diseases further comprises the Rad50, Smg6, and Mre11 markers and at least one marker comprising a polypeptide, fragment, variants, analogs and mutants thereof of Rad50.

In another preferred embodiment, a biomarker composition for the prognosis of recovery from heart failure and cardiac diseases comprising markers Rad50, Smg6, and Mre11 and combinations thereof. In one aspect, the biomarker further comprises a marker selected from the group consisting of Mre11, Nbs1, Mre11/Rad50/Nbs1 complex, enzymes and t-loop generation factors.

In another preferred embodiment, the biomarker composition for the prognostic recovery from heart failure and cardiac diseases comprises the Rad50, Smg6, and Mre11 markers and at least one marker comprising a polynucleotide, fragments, alleles, variants, mutants, complementary sequences thereof of Rad50, Smg6, and Mre11.

In another preferred embodiment, the biomarker composition for the prognosis of recovery from heart failure and cardiac diseases further comprises the Rad50, Smg6, and Mre11 markers and at least one marker comprising a polypeptide, fragment, variants, analogs and mutants thereof of Rad50, Smg6, and Mre11.

In another preferred embodiment, the biomarker composition for the prognosis of recovery from heart failure and cardiac diseases further comprises the Rad50, Smg6, and Mre11 markers and at least one marker comprising a polynucleotide, fragments, alleles, variants, mutants substituted polynucleotides and complementary sequences thereof of Smg6.

In another preferred embodiment, the biomarker composition for the prognosis of recovery from heart failure and cardiac diseases further comprises the Rad50, Smg6, and Mre11 markers and at least one marker comprising a polypeptide, fragment, variants, analogs and mutants thereof of Smg6.

In another preferred embodiment, the biomarker composition for the prognosis of recovery from heart failure and cardiac diseases further comprises the Rad50, Smg6, and Mre11 markers and at least one marker comprising a polynucleotide, fragments, alleles, variants, mutants, substituted polynucleotides and complementary sequences thereof of Rad50.

In another preferred embodiment, the biomarker composition for the prognosis of recovery from heart failure and cardiac diseases further comprises the Rad50, Smg6, and Mre11 markers and at least one marker comprising a polypeptide, fragment, variants, analogs and mutants thereof of Rad50.

In a preferred embodiment, oligonucleotides comprising Rad50, Smg6, and Mre11 are used in a variety of diagnostic or prognostic assays. For example, the sequences can be radiolabeled to identify hybridization, use of the primers in PCR, generation of peptides, aptamers and antibodies directed to the desired sequences, etc.

In one embodiment, a method of diagnosing heart failure and other cardiac disorders or diseases, or prognosis of recovery from heart failure and other cardiac diseases or disorders comprises obtaining a biological sample from a patient; identifying Rad50, Smg6, and Mre11, combinations or a portion thereof; detecting the presence, absence or variation in concentration of a peptide encoded by Rad50, Smg6, and Mre11, mutants, variants, alleles, complementary sequence and fragments thereof; comparing the concentrations of the peptide between a normal individual, a survivor and the patient.

The biological samples used in the present invention can include cells, protein or membrane extracts of cells, blood or biological fluids such as ascites fluid, lymphatic fluids, brain fluid (e.g., cerebrospinal fluid). Examples of solid biological samples include, but are not limited to, samples taken from tissues of the central nervous system, bone, breast, kidney, cervix, endometrium, head/neck, gallbladder, parotid gland, prostate, pituitary gland, muscle, esophagus, stomach, small intestine, colon, liver, spleen, pancreas, thyroid, heart, lung, bladder, adipose, lymph node, uterus, ovary, adrenal gland, testes, tonsils and thymus. Examples of “body fluid samples” include, but are not limited to blood, serum, semen, prostate fluid, seminal fluid, urine, saliva, sputum, mucus, bone marrow, lymph, and tears.

In another preferred embodiment, the patient or individual is a mammal. This includes humans of any age. For example, an embryo, neonate, infant, child, teenager or adult.

In another preferred embodiment, an Rad50, Smg6, and Mre11 peptide or nucleic acid is identified by an antibody or aptamer.

In another preferred embodiment, a kit comprises primers of Rad50, Smg6, and Mre11; thermostable polymerase, and A, G, C, T nucleotides. The primers can be selected from any nucleic acid region 5′ to the coding region of each molecule, 3′ to the coding region or any region of the molecule selected by one of ordinary skill in the art.

In another preferred embodiment, a kit comprises at least one of Rad50, Smg6, and Mre11, peptides thereof, or antibodies specific for Rad50, Smg6, and Mre11 or peptides thereof.

In some instances, such as when unusually small amounts of RNA are recovered and only small amounts of cDNA are generated therefrom, it is desirable or necessary to perform a PCR reaction on the first PCR reaction product. That is, if difficult to detect quantities of amplified DNA are produced by the first reaction, a second PCR can be performed to make multiple copies of DNA sequences of the first amplified DNA. A nested set of primers are used in the second PCR reaction. The nested set of primers hybridize to sequences downstream of the 5′ primer and upstream of the 3′ primer used in the first reaction.

According to the invention, diagnostic kits can be assembled which is useful to practice methods of detecting the presence of mRNA or cDNA that encodes at least one of Rad50, Smg6, and Mre11 in tissue samples. Such diagnostic kits comprise oligonucleotides which are useful as primers for performing PCR methods. It is preferred that diagnostic kits according to the present invention comprise a container comprising a size marker to be run as a standard on a gel used to detect the presence of amplified DNA. The size marker is the same size as the DNA generated by the primers in the presence of the mRNA or cDNA encoding Rad50, Smg6, and Mre11 or combinations thereof.

In another preferred embodiment, a kit comprises reagents for identifying and measuring the levels of Rad50, Smg6, and Mre11 using real-time PCR (RT-PCR). The kit can include one or more of primers suitable for hybridizing to Rad50, Smg6, and Mre11.

Another method of determining whether a sample contains cells expressing at least one of Rad50, Smg6, and Mre11, is by Northern blot analysis of mRNA extracted from a tissue sample. The techniques for performing Northern blot analyses are well known by those having ordinary skill in the art and are described in Sambrook, J. et al., Molecular Cloning: A Laboratory Manual, (1989) Cold Spring Harbor Laboratory Press, Cold Spring Harbor, N.Y. mRNA extraction, electrophoretic separation of the mRNA, blotting, probe preparation and hybridization are all well known techniques that can be routinely performed using readily available starting material.

One having ordinary skill in the art, performing routine techniques, could design probes to identify mRNA encoding at least one of Rad50, Smg6, and Mre11, using their sequence information. The mRNA is extracted using poly dT columns and the material is separated by electrophoresis and, for example, transferred to nitrocellulose paper. Labeled probes made from an isolated specific fragment or fragments can be used to visualize the presence of a complementary fragment fixed to the paper.

According to the invention, diagnostic kits can be assembled which is useful to practice methods of detecting the presence of mRNA that encodes at least one of Rad50, Smg6, and Mre11, in tissue samples by Northern blot analysis. Such diagnostic kits comprise oligonucleotides which are useful as probes for hybridizing to the mRNA. The probes may be radiolabeled. It is preferred that diagnostic kits according to the present invention comprise a container comprising a size marker to be run as a standard on a gel. It is preferred that diagnostic kits according to the present invention comprise a container comprising a positive control which will hybridize to the probe.

Another method of detecting the presence of mRNA encoding at least one of Rad50, Smg6, and Mre11, protein is by oligonucleotide hybridization technology. Oligonucleotide hybridization technology is well known to those having ordinary skill in the art. Briefly, detectable probes which contain a specific nucleotide sequence that will hybridize to nucleotide sequence of mRNA encoding Rad50, Smg6, or Mre11, protein. RNA or cDNA made from RNA from a sample is fixed, usually to filter paper or the like. The probes are added and maintained under conditions that permit hybridization only if the probes fully complement the fixed genetic material. The conditions are sufficiently stringent to wash off probes in which only a portion of the probe hybridizes to the fixed material. Detection of the probe on the washed filter indicates complementary sequences. One having ordinary skill in the art, using the sequence information of Rad50, Smg6, and Mre11, can design probes which are fully complementary to mRNA sequences but not genomic DNA sequences. Hybridization conditions can be routinely optimized to minimize background signal by non-fully complementary hybridization.

The present invention includes labeled oligonucleotides which are useful as probes for performing oligonucleotide hybridization. That is, they are fully complementary with mRNA sequences but not genomic sequences. For example, the mRNA sequence includes portions encoded by different exons. The labeled probes of the present invention are labeled with radiolabeled nucleotides or are otherwise detectable by readily available nonradioactive detection systems.

According to the invention, diagnostic kits can be assembled which is useful to practice oligonucleotide hybridization methods of the invention. Such diagnostic kits comprise a labeled oligonucleotide which encodes portions of at least one of Rad50, Smg6, and Mre11, encoded by different exons. It is preferred that labeled probes of the oligonucleotide diagnostic kits according to the present invention are labeled with a radionucleotide. The oligonucleotide hybridization-based diagnostic kits according to the invention preferably comprise DNA samples that represent positive and negative controls. A positive control DNA sample is one that comprises a nucleic acid molecule which has a nucleotide sequence that is fully complementary to the probes of the kit such that the probes will hybridize to the molecule under assay conditions. A negative control DNA sample is one that comprises at least one nucleic acid molecule, the nucleotide sequence of which is partially complementary to the sequences of the probe of the kit. Under assay conditions, the probe will not hybridize to the negative control DNA sample.

Another aspect of the invention relates to methods of analyzing tissue samples which are fixed sections routinely prepared by surgical pathologists to characterize and evaluate cells. In some embodiments, the cells are from heart tissue and are analyzed to determine and evaluate the extent of at least one of Rad50, Smg6, and Mre11, expression.

The present invention relates to in vitro kits for evaluating tissues samples to determine the level of at least one of Rad50, Smg6, and Mre11, expression and to reagents and compositions useful to practice the same. The tissue is analyzed to identify the presence or absence of the at least one of Rad50, Smg6, and Mre11, protein. Techniques such as binding assays, e.g. Rad50/anti-Rad50 binding assays, and immunohistochemistry assays may be performed to determine whether at least one of Rad50, Smg6, and Mre11, is absent in cells in the tissue sample which are indicative of poor prognosis of recovery and long term survival from heart failure and other cardiac disorders or upregulated which is indicative and diagnostic of recovery and long term survival from heart failure and other cardiac disorders. The presence of mRNA that encodes the at least one of Rad50, Smg6, and Mre11 protein or cDNA generated therefrom can be determined using techniques such as in situ hybridization, immunohistochemistry.

In situ hybridization technology is well known by those having ordinary skill in the art. Briefly, cells are fixed and detectable probes which contain a specific nucleotide sequence are added to the fixed cells. If the cells contain complementary nucleotide sequences, the probes, which can be detected, will hybridize to them. One having ordinary skill in the art, using the sequence information of Rad50, Smg6, or Mre11 can design probes useful in in situ hybridization technology to identify cells that express at least one of Rad50, Smg6, and Mre11.

For in situ hybridization according to the invention, it is preferred that the probes are detectable by fluorescence. A common procedure is to label probe with biotin-modified nucleotide and then detect with fluorescently-tagged avidin. Hence, the probe does not itself have to be labeled with florescent but can be subsequently detected with florescent marker.

Cells are fixed and the probes are added to the genetic material. Probes will hybridize to the complementary nucleic acid sequences present in the sample. Using a fluorescent microscope, the probes can be visualized by their fluorescent markers.

According to the invention, diagnostic kits can be assembled which is useful to practice in situ hybridization methods of the invention are fully complementary with mRNA sequences but not genomic sequences. For example, the mRNA sequence includes portions encoded by different exons. It is preferred that labeled probes of the in situ diagnostic kits according to the present invention are labeled with a fluorescent marker.

Immunohistochemistry techniques may be used to identify and essentially stain cells which express at least one of Rad50, Smg6, and Mre11. Anti-marker, e.g. anti-Rad50, antibodies are contacted with fixed cells and the Rad50 present in the cells reacts with the antibodies. The antibodies are detectably labeled or detected using labeled second antibody or protein A to stain the cells.

According to some embodiments, diagnostic reagents and kits are provided for performing immunoassays to determine the presence or absence of at least one of Rad50, Smg6, and Mre11 protein or fragments thereof in a sample from an individual. Kits may additionally include one or more of the following: means for detecting antibodies bound to at least one of Rad50, Smg6, and Mre11 present in a sample, instructions for performing the method, and diagrams or photographs that are representative of how positive and/or negative results appear. In addition, kits may comprise optional positive controls such as at least one of Rad50, Smg6, and Mre11 protein. Further, optional negative controls may be provided.

Immunoassay methods may be used to identify individuals identifying and measuring the levels of Rad50, Smg6, and Mre11 by detecting the absence or deficiency of at least one of Rad50, Smg6, and Mre11 in sample of tissue or body fluid using antibodies which bind to Rad50, Smg6, or Mre11. The antibodies are preferably monoclonal antibodies. The antibodies are preferably raised against FMR4 made in human cells. Immunoassays are well known and there design may be routinely undertaken by those having ordinary skill in the art. The techniques for producing monoclonal antibodies are outlined in Harlow, E. and D. Lane (1988) Antibodies: A Laboratory Manual, Cold Spring Harbor Laboratory, Cold Spring Harbor, N.Y., which is incorporated herein by reference, provide detailed guidance for the production of hybridomas and monoclonal antibodies which specifically bind to Rad50, Smg6, or Mre11.

According to some embodiments, immunoassays comprise allowing proteins in the sample to bind a solid phase support such as a plastic surface. Detectable antibodies are then added which selectively binding to Rad50, Smg6, or Mre11. Detection of the detectable antibody indicates the presence of Rad50, Smg6, or Mre11. The detectable antibody may be a labeled or an unlabelled antibody. Unlabelled antibody may be detected using a second, labeled antibody that specifically binds to the first antibody or a second, unlabelled antibody which can be detected using labeled protein A, a protein that complexes with antibodies. Various immunoassay procedures are described in Immunoassays for the 80's, Voller, et al., Ed., University Park, 1981, which is incorporated herein by reference.

Simple immunoassays may be performed in which a solid phase support is contacted with the test sample. Any proteins present in the test sample bind the solid phase support and can be detected by a specific, detectable antibody preparation. Such a technique is the essence of the dot blot, Western blot and other such similar assays.

Other immunoassays may be more complicated but actually provide excellent results. Typical and preferred immunometric assays include “forward” assays for the detection of a protein in which a first anti-protein antibody bound to a solid phase support is contacted with the test sample. After a suitable incubation period, the solid phase support is washed to remove unbound protein. A second, distinct anti-protein antibody is then added which is specific for a portion of the specific protein not recognized by the first antibody. The second antibody is preferably detectable. After a second incubation period to permit the detectable antibody to complex with the specific protein bound to the solid phase support through the first antibody, the solid phase support is washed a second time to remove the unbound detectable antibody. Alternatively, the second antibody may not be detectable. In this case, a third detectable antibody, which binds the second antibody is added to the system. This type of “forward sandwich” assay may be a simple yes/no assay to determine whether binding has occurred or may be made quantitative by comparing the amount of detectable antibody with that obtained in a control. Such “two-site” or “sandwich” assays are described by Wide, Radioimmune Assay Method, (1970) Kirkham, Ed., E. & S. Livingstone, Edinburgh, pp. 199-206, which is incorporated herein by reference.

Other types of immunometric assays are the so-called “simultaneous” and “reverse” assays. A simultaneous assay involves a single incubation step wherein the first antibody bound to the solid phase support, the second, detectable antibody and the test sample are added at the same time. After the incubation is completed, the solid phase support is washed to remove unbound proteins. The presence of detectable antibody associated with the solid support is then determined as it would be in a conventional “forward sandwich” assay. The simultaneous assay may also be adapted in a similar manner for the detection of antibodies in a test sample.

The “reverse” assay comprises the stepwise addition of a solution of detectable antibody to the test sample followed by an incubation period and the addition of antibody bound to a solid phase support after an additional incubation period. The solid phase support is washed in conventional fashion to remove unbound protein/antibody complexes and unreacted detectable antibody. The determination of detectable antibody associated with the solid phase support is then determined as in the “simultaneous” and “forward” assays. The reverse assay may also be adapted in a similar manner for the detection of antibodies in a test sample.

The “Rad50” marker is used throughout as merely exemplary and does not limit the description or embodiments to just Rad50, but includes Smg6, Mre11, the MRN complex, fragments, mutants variants and complementary sequences thereof. The first component of the immunometric assay may be added to nitrocellulose or other solid phase support which is capable of immobilizing proteins. The first component for determining the presence of Rad50 in a test sample is anti-Rad50 antibody. By “solid phase support” or “support” is intended any material capable of binding proteins. Well-known solid phase supports include glass, polystyrene, polypropylene, polyethylene, dextran, nylon, amylases, natural and modified celluloses, polyacrylamides, agaroses, and magnetite. The nature of the support can be either soluble to some extent or insoluble for the purposes of the present invention. The support configuration may be spherical, as in a bead, or cylindrical, as in the inside surface of a test tube or the external surface of a rod. Alternatively, the surface may be flat such as a sheet, test strip, etc. Those skilled in the art will know many other suitable “solid phase supports” for binding proteins or will be able to ascertain the same by use of routine experimentation. A preferred solid phase support is a 96-well microtiter plate.

To detect the presence of Rad50, detectable anti-Rad50 antibodies are used. Several methods are well known for the detection of antibodies. One method in which the antibodies can be detectably labeled is by linking the antibodies to an enzyme and subsequently using the antibodies in an enzyme immunoassay (EIA) or enzyme-linked immunosorbent assay (ELISA), such as a capture ELISA. The enzyme, when subsequently exposed to its substrate, reacts with the substrate and generates a chemical moiety which can be detected, for example, by spectrophotometric, fluorometric or visual means. Enzymes which can be used to detectably label antibodies include, but are not limited to malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. One skilled in the art would readily recognize other enzymes which may also be used.

Another method in which antibodies can be detectably labeled is through radioactive isotopes and subsequent use in a radioimmunoassay (RIA) (see, for example, Work, et al., Laboratory Techniques and Biochemistry in Molecular Biology, North Holland Publishing Company, N.Y., 1978, which is incorporated herein by reference). The radioactive isotope can be detected by such means as the use of a gamma counter or a scintillation counter or by autoradiography. Isotopes which are particularly useful for the purpose of the present invention are ³H, ¹²⁵I, ¹³¹I, ³⁵S, and ¹⁴C. One skilled in the art would readily recognize other radioisotopes which may also be used.

It is also possible to label the antibody with a fluorescent compound. When the fluorescent-labeled antibody is exposed to light of the proper wavelength, its presence can be detected due to its fluorescence. Among the most commonly used fluorescent labeling compounds are fluorescein isothiocyanate, rhodamine, phycoerythrin, phycocyanin, allophycocyanin, o-phthaldehyde and fluorescamine. One skilled in the art would readily recognize other fluorescent compounds which may also be used.

Antibodies can also be detectably labeled using fluorescence-emitting metals such as ¹⁵²Eu, or others of the lanthanide series. These metals can be attached to the protein-specific antibody using such metal chelating groups as diethylenetriaminepentaacetic acid (DTPA) or ethylenediamine-tetraacetic acid (EDTA). One skilled in the art would readily recognize other fluorescence-emitting metals as well as other metal chelating groups which may also be used.

Antibodies can also be detectably labeled by coupling to a chemiluminescent compound. The presence of the chemiluminescent-labeled antibody is determined by detecting the presence of luminescence that arises during the course of a chemical reaction. Examples of particularly useful chemoluminescent labeling compounds are luminol, isoluminol, theromatic acridinium ester, imidazole, acridinium salt and oxalate ester. One skilled in the art would readily recognize other chemiluminescent compounds which may also be used.

Likewise, a bioluminescent compound may be used to label antibodies. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin. One skilled in the art would readily recognize other bioluminescent compounds which may also be used.

Detection of the protein-specific antibody, fragment or derivative may be accomplished by a scintillation counter if, for example, the detectable label is a radioactive gamma emitter. Alternatively, detection may be accomplished by a fluorometer if, for example, the label is a fluorescent material. In the case of an enzyme label, the detection can be accomplished by colorometric methods which employ a substrate for the enzyme. Detection may also be accomplished by visual comparison of the extent of enzymatic reaction of a substrate in comparison with similarly prepared standards. One skilled in the art would readily recognize other appropriate methods of detection which may also be used.

The binding activity of a given lot of antibodies may be determined according to well known methods. Those skilled in the art will be able to determine operative and optimal assay conditions for each determination by employing routine experimentation.

In a preferred embodiment, the kits include antibodies for detection of Rad50, Smg6, Mre11, Nbs1, or the Mre11/Rad50/Nbs1 complex and identifying variants thereof, using Western blot analysis.

Positive and negative controls may be performed in which known amounts of Rad50 and no Rad50, respectively, are added to assays being performed in parallel with the test assay. One skilled in the art would have the necessary knowledge to perform the appropriate controls.

An “antibody composition” refers to the antibody or antibodies required for the detection of the protein. For example, the antibody composition used for the detection of Rad50 in a test sample comprises a first antibody which binds Rad50, as well as a second or third detectable antibody that binds the first or second antibody, respectively.

To examine a test sample for the presence or absence of Rad50, a standard immunometric assay such as the one described herein may be performed. A first anti-Rad50 antibody, which recognizes, for example, a specific portion of Rad50 is added to a 96-well microtiter plate in a volume of buffer. The plate is incubated for a period of time sufficient for binding to occur and subsequently washed with PBS to remove unbound antibody. The plate is then blocked with a PBS/BSA solution to prevent sample proteins from non-specifically binding the microtiter plate. Test sample are subsequently added to the wells and the plate is incubated for a period of time sufficient for binding to occur. The wells are washed with PBS to remove unbound protein. Labeled anti-Rad50 antibodies, which recognize portions of Rad50 not recognized by the first antibody, are added to the wells. The plate is incubated for a period of time sufficient for binding to occur and subsequently washed with PBS to remove unbound, labeled anti-Rad50 antibody. The amount of labeled and bound anti-Rad50 antibody is subsequently determined by standard techniques.

Kits which are useful for the detection of, for example, Rad50 in a test sample comprise a container comprising anti-Rad50 antibodies and a container or containers comprising controls. Controls include one control sample which does not contain Rad50 and/or another control sample which contained Rad50. The anti-Rad50 antibodies used in the kit are detectable such as being detectably labeled. If the detectable anti-Rad50 antibody is not labeled, it may be detected by second antibodies or protein A, for example, which may also be provided in some kits in separate containers. Additional components in some kits include solid support, buffer, and instructions for carrying out the assay. The immunoassay is useful for detecting Rad50 in homogenized tissue samples and body fluid samples including the plasma portion or cells in the fluid sample.

As discussed infra, in all of the embodiments, the detection can also include other antibodies such as those detecting Rad50, Smg6, Mre11, Nbs1, or the Mre11/Rad50/Nbs1 complex, fragments, mutants variants and complementary sequences thereof etc, nucleic acids, peptides, for example, as a control, as part of the detection and the like.

Western blots may be used in methods of identifying individuals who will recover and survive heart failure and other cardiac disorders, by detecting presence of Rad50, Smg6, Mre11, Nbs1, or the Mre11/Rad50/Nbs1 complex in samples of tissue, such as for example, heart. Western blots use detectable anti-Rad50 antibodies to bind to any Rad50 present in a sample and thus indicate the presence of the protein in the sample. Western blot techniques, which are described in Sambrook, J. et al., (1989) Molecular Cloning: A Laboratory Manual, Cold Spring Harbor Laboratory Press, Cold Spring Harbor, N.Y., which is incorporated herein by reference, are similar to immunoassays with the essential difference being that prior to exposing the sample to the antibodies, the proteins in the samples are separated by gel electrophoresis and the separated proteins are then probed with antibodies. In some preferred embodiments, the matrix is an SDS-PAGE gel matrix and the separated proteins in the matrix are transferred to a carrier such as filter paper prior to probing with antibodies.

Anti-Rad50 antibodies described above are useful in Western blot methods. Generally, samples are homogenized and cells are lysed using detergent such as Triton-X. The material is then separated by the standard techniques in Sambrook, J. et al., (1989) Molecular Cloning: A Laboratory Manual, Cold Spring Harbor Laboratory Press, Cold Spring Harbor, N.Y.

Kits which are useful for the detection of Rad50, Smg6, Mre11, Nbs1, or the Mre11/Rad50/Nbs1 complex in a test sample by Western blot comprise a container comprising Rad50, Smg6, Mre11, Nbs1, or the Mre11/Rad50/Nbs1 complex antibodies and a container or containers comprising controls. For example, controls include one control sample which does not contain Rad50 and/or another control sample which contained Rad50. The anti-Rad50 antibodies used in the kit are detectable such as being detectably labeled. If the detectable anti-Rad50 is not labeled, it may be detected by second antibodies or protein A for example which may also be provided in some kits in separate containers. Additional components in some kits include instructions for carrying out the assay. The means to detect anti-Rad50 antibodies that are bound to Rad50 include the immunoassays described above.

Aspects of the present invention also include various methods of determining whether a sample contains cells that express Rad50, Smg6, Mre11, Nbs1, or the Mre11/Rad50/Nbs1 complex by sequence-based molecular analysis. Several different methods are available for doing so including those using Polymerase Chain Reaction (PCR) technology, using Northern blot technology, oligonucleotide hybridization technology, and in situ hybridization technology. According to the invention, samples are screened to determine the presence or absence of mRNA that encodes Rad50, Smg6, Mre11, Nbs1, or the Mre11/Rad50/Nbs1 complex.

The invention also relates to oligonucleotide probes and primers used in the methods of identifying mRNA that encodes Rad50, Smg6, Mre11, Nbs1, or the Mre11/Rad50/Nbs1 complex and to diagnostic kits which comprise such components. The mRNA sequence-based methods for determining whether a sample mRNA encoding Rad50, Smg6, Mre11, Nbs1, or the Mre11/Rad50/Nbs1 complex include but are not limited to PCR technology, Northern and Southern blot technology, in situ hybridization technology and oligonucleotide hybridization technology.

### Treatment

Treatment is an intervention performed with the intention of preventing the development or altering the pathology or symptoms of a disorder. Accordingly, treatment refers to both therapeutic treatment and prophylactic or preventative measures. Treatment may also be specified as palliative care. Those in need of treatment include those already with the disorder as well as those in which the disorder is to be prevented.

In a preferred embodiment, the compositions of the invention are administered to patients for the treatment of heart failure, ischemic heart injury and other cardiac disorders. The compositions comprise agents which increase expression of Rad50, Smg6, Mre11, Nbs1, or the Mre11/Rad50/Nbs1 complex, fragments, mutants, variants and complementary sequences thereof; cells, e.g. stem cells expressing at least one of Rad50, Smg6, Mre11, Nbs1, or the Mre11/Rad50/Nbs1 complex, fragments, mutants, variants and complementary sequences thereof; vectors encoding at least one of Rad50, Smg6, Mre11, Nbs1, or the Mre11/Rad50/Nbs1 complex, fragments, mutants, variants and complementary sequences thereof; polynucleotides and/or polypeptides of Rad50, Smg6, Mre11, Nbs1, or the Mre11/Rad50/Nbs1 complex, fragments, mutants, variants and complementary sequences thereof.

Rad50 will be used throughout for illustrative purposes only and is not meant to be construed as being limited to Rad50, and includes all of the compositions described herein, e.g. Rad50, Smg6, Mre11, Nbs1, or the Mre11/Rad50/Nbs1 complex, fragments, mutants, variants and complementary sequences thereof.

In one embodiment, the compositions are administered as a replacement therapy. Peptides of Rad50 can be administered to a patient, for example, in the form of a peptide in a pharmaceutical compositions, as a vector expressing Rad50, a peptide of Rad50 and the like.

In another embodiment, Rad50 expression can be disrupted, modulated, increased, decreased, silenced by antibodies specific to Rad50, siRNA, antisense oligonucleotides, small molecule inhibition of Rad50 peptides and the like. Preferably, Rad50 expression is modulated to increase the levels to those levels found in normal individuals.

If disruption is desired, the disruption of a desired target nucleic acid can be carried out in several ways known in the art. For example, siRNA. Enzymatic nucleic acid molecules (e.g., ribozymes) are nucleic acid molecules capable of catalyzing one or more of a variety of reactions, including the ability to repeatedly cleave other separate nucleic acid molecules in a nucleotide base sequence-specific manner. Such enzymatic nucleic acid molecules can be used, for example, to target virtually any RNA transcript (Zaug et al., 324, Nature 429 1986; Cech, 260 JAMA 3030, 1988; and Jefferies et al., 17 Nucleic Acids Research 1371, 1989). Because of their sequence-specificity, trans-cleaving enzymatic nucleic acid molecules show promise as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the mRNA non-functional and abrogates protein expression from that RNA. In this manner, synthesis of a protein associated with a disease state can be selectively inhibited.

In general, enzymatic nucleic acids with RNA cleaving activity act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Several approaches such as in vitro selection (evolution) strategies (Orgel, 1979, Proc. R. Soc. London, B 205, 435) have been used to evolve new nucleic acid catalysts capable of catalyzing a variety of reactions, such as cleavage and ligation of phosphodiester linkages and amide linkages, (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al., 1993, Science 261:1411-1418; Szostak, 1993, TIBS 17, 89-93; Kumar et al., 1995, FASEB J., 9, 1183; Breaker, 1996, Curr. Op. Biotech., 7, 442).

The development of ribozymes that are optimal for catalytic activity would contribute significantly to any strategy that employs RNA-cleaving ribozymes for the purpose of regulating gene expression. The hammerhead ribozyme, for example, functions with a catalytic rate (kcat) of about 1 min-1 in the presence of saturating (10 mM) concentrations of Mg²⁺ cofactor. An artificial “RNA ligase” ribozyme has been shown to catalyze the corresponding self-modification reaction with a rate of about 100 min−1. In addition, it is known that certain modified hammerhead ribozymes that have substrate binding arms made of DNA catalyze RNA cleavage with multiple turn-over rates that approach 100 min−1. Finally, replacement of a specific residue within the catalytic core of the hammerhead with certain nucleotide analogues gives modified ribozymes that show as much as a 10-fold improvement in catalytic rate. These findings demonstrate that ribozymes can promote chemical transformations with catalytic rates that are significantly greater than those displayed in vitro by most natural self-cleaving ribozymes. It is then possible that the structures of certain self-cleaving ribozymes may be optimized to give maximal catalytic activity, or that entirely new RNA motifs can be made that display significantly faster rates for RNA phosphodiester cleavage.

Intermolecular cleavage of an RNA substrate by an RNA catalyst that fits the “hammerhead” model was first shown in 1987 (Uhlenbeck, O. C. (1987) Nature, 328: 596-600). The RNA catalyst was recovered and reacted with multiple RNA molecules, demonstrating that it was truly catalytic.

Catalytic RNAs designed based on the “hammerhead” motif have been used to cleave specific target sequences by making appropriate base changes in the catalytic RNA to maintain necessary base pairing with the target sequences (Haseloff and Gerlach, Nature, 334, 585 (1988); Walbot and Bruening, Nature, 334, 196 (1988); Uhlenbeck, O. C. (1987) Nature, 328: 596-600; Koizumi, M., Iwai, S, and Ohtsuka, E. (1988) FEBS Lett., 228: 228-230). This has allowed use of the catalytic RNA to cleave specific target sequences and indicates that catalytic RNAs designed according to the “hammerhead” model may possibly cleave specific substrate RNAs in vivo. (see Haseloff and Gerlach, Nature, 334, 585 (1988); Walbot and Bruening, Nature, 334, 196 (1988); Uhlenbeck, O. C. (1987) Nature, 328: 596-600).

RNA interference (RNAi) has become a powerful tool for blocking gene expression in mammals and mammalian cells. This approach requires the delivery of small interfering RNA (siRNA) either as RNA itself or as DNA, using an expression plasmid or virus and the coding sequence for small hairpin RNAs that are processed to siRNAs. This system enables efficient transport of the pre-siRNAs to the cytoplasm where they are active and permit the use of regulated and tissue specific promoters for gene expression.

Peptides/Polypeptides: In some embodiments, the peptides used in treatment include peptides of Rad50, Smg6, Mre11, Nbs1, or the Mre11/Rad50/Nbs1 complex, fragments, mutants, variants and complementary sequences thereof.

In some embodiments, the compositions comprise a polypeptide that is at least about 85% identical to the amino acid sequence of Rad50, Smg6, Mre11, Nbs1, or the Mre11/Rad50/Nbs1 complex, fragments, mutants, variants and complementary sequences thereof. In some embodiments, the polypeptide is at least about 90%, 95%, 99%, or 100% identical to the full length sequence of Rad50, Smg6, Mre11, Nbs1, or the Mre11/Rad50/Nbs1 complex, fragments, mutants, variants and complementary sequences thereof. In some embodiments, the peptide is at least about 10, 12, 14, 16, 18, 20, 22, 24, 26, 28, 30, 32, 34, 36, 38, 39, 40, 45, 50, 55, 60, 65, 70, 75, 80 or more amino acids long. A “polypeptide comprising a fragment of Rad50, Smg6, Mre11, Nbs1, or the Mre11/Rad50/Nbs1 complex, fragments, mutants, variants and complementary sequences thereof” includes less than the full length of each, but can include other (e.g., non-Rad50 proteins or fragments thereof, e.g., fluorescent proteins such as green fluorescent protein (GFP), red fluorescent protein (RFP), blue fluorescent protein (BFP) or yellow fluorescent protein (YFP), or peptides that enhance delivery, e.g., a TAT protein transduction domain (PTD).

The term “isolated polypeptide” used here in means a polypeptide that is substantially pure and free from other biological macromolecules. The substantially pure polypeptide is at least 75% (e.g., at least 80, 85, 95, or 99%) pure by dry weight. Purity can be measured by any appropriate standard method, for example by column chromatography, polyacrylamide gel electrophoresis, or HPLC analysis.

The polypeptides of the present invention includes variants of Rad50, Smg6, Mre11, Nbs1, or the Mre11/Rad50/Nbs1 complex peptides as long as the variants are at least 50% identical the native or wild-type peptides. The variants may be a polypeptide comprising the amino acid sequence of, for example, Rad50 peptides in which one or more amino acids have been substituted, deleted, added, and/or inserted. The variants may also be a polypeptide encoded by a nucleic acid comprising a strand that hybridizes under high stringent conditions to a nucleotide sequence consisting of, for example, Rad50.

Polypeptides having amino acid sequences modified by deleting, adding and/or replacing one or more amino acid residues of a certain amino acid sequence, have been known to retain the original biological activity (Mark, D. F. et al., Proc. Natl. Acad. Sci. USA (1984) 81, 5662-5666, Zoller, M. J. & Smith, M., Nucleic Acids Research (1982) 10, 6487-6500, Wang, A. et al., Science 224, 1431-1433, Dalbadie-McFarland, G. et al., Proc. Natl. Acad. Sci. USA (1982) 79, 6409-6413).

The number of amino acids that are mutated by substitution, deletion, addition, and/or insertion is not particularly restricted. Normally, it is 20% or less, preferably 15% or less, and more preferably 10% or less of the total amino acid residues.

As for the amino acid residue to be mutated, it is preferable to be mutated into a different amino acid in which the properties of the amino acid side-chain are conserved. Examples of properties of amino acid side chains are, hydrophobic amino acids (A, I, L, M, F, P, W, Y, V), hydrophilic amino acids (R, D, N, C, E, Q, G, H, K, S, T), and amino acids comprising the following side chains: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); abase containing side-chain (R, K, H); and an aromatic containing side-chain (H, F, Y, W) (The parenthetic letters indicate the one-letter codes of amino acids). A “conservative amino acid substitution” is a replacement of one amino acid belonging to one of the above groups with another amino acid in the same group.

A deletion variant includes a fragment of the amino acid sequence encoded by, for example, Rad50 polynucleotide. The fragment is a polypeptide having an amino acid sequence which is partly, but not entirely, identical to the above polypeptides of this invention. The polypeptide fragments of this invention usually consist of 8 amino acid residues or more, and preferably 12 amino acid residues or more (for example, 15 amino acid residues or more). Examples of preferred fragments include truncation polypeptides, having amino acid sequences lacking a series of amino acid residues including either the amino terminus or carboxyl terminus, or two series of amino acid residues, one including the amino terminus and the other including the carboxyl terminus. Furthermore, fragments featured by structural or functional characteristics are also preferable, which include those having α-helix and α-helix forming regions, β-sheet and β-sheet forming regions, turn and turn forming regions, coil and coil forming regions, hydrophilic regions, hydrophobic regions, α-amphipathic regions, β-amphipathic regions, variable regions, surface forming regions, substrate-binding regions, and high antigenicity index region. Biologically active fragments are also preferred. Biologically active fragments mediate the activities of the polypeptides of this invention, which fragments include those having similar or improved activities, or reduced undesirable activities. For example, fragments having the activity to transduce signals into cells via binding of a ligand, and furthermore, fragments having antigenicity or immunogenicity in animals, especially humans are included. These polypeptide fragments preferably retain the antigenicity of the polypeptides of this invention.

Further, an addition variant includes a fusion protein of the polypeptide of the present invention and another peptide or polypeptide. Fusion proteins can be made by techniques well known to a person skilled in the art, such as by linking the DNA encoding the polypeptide of the invention with DNA encoding other peptides or polypeptides, so as the frames match, inserting this into an expression vector and expressing it in a host. There is no restriction as to the peptides or polypeptides fused to the polypeptide of the present invention.

Known peptides, for example, FLAG (Hopp, T. P. et al., Biotechnology (1988) 6, 1204-1210), 6× His containing six His (histidine) residues, 10×His, Influenza agglutinin (HA), human c-myc fragment, VSP-GP fragment, p18HIV fragment, T7-tag, HSV-tag, E-tag, SV40T antigen fragment, lck tag, α-tubulin fragment, B-tag, Protein C fragment, fluorescent proteins such as green fluorescent protein (GFP), red fluorescent protein (RFP), blue fluorescent protein (BFP) or yellow fluorescent protein (YFP), or peptides that enhance delivery, e.g., a TAT protein transduction domain (PTD) and such, can be used as peptides that are fused to the polypeptide of the present invention.

Fusion proteins can be prepared by fusing commercially available DNA encoding these peptides or polypeptides with the DNA encoding the polypeptide of the present invention and expressing the fused DNA prepared.

The variant polypeptide is preferably at least 65% identical to the amino acid sequence of native or wild-type Rad50, Smg6, Mre11, Nbs1, or the Mre11/Rad50/Nbs1 complex. More specifically, the modified polypeptide is at least 65%, 70%, 75%, 80%, 85%, 90%, 95%, 96%, 97%, 98%, 99%, or more, identical to the amino acid sequence encoded by Rad50, Smg6, Mre11, Nbs1, or the Mre11/Rad50/Nbs1 complex. In the case of a modified polypeptide which is longer than or equivalent in length to the reference sequence, e.g., the amino acid sequence of Rad50, the comparison is made with the full length of the reference sequence. Where the modified polypeptide is shorter than the reference sequence, e.g., the amino acid sequence of Rad50, the comparison is made to segment of the reference sequence of the same length.

The polypeptide of the present invention can be prepared by methods known to one skilled in the art, as a natural polypeptide or a recombinant polypeptide made using genetic engineering techniques as described above. For example, a natural polypeptide can be obtained by preparing a column coupled with an antibody obtained by immunizing a small animal with the recombinant polypeptide, and performing affinity chromatography from extracts of heart tissues or cells expressing high levels of the polypeptide of the present invention. A recombinant polypeptide can be prepared by inserting DNA encoding the polypeptide of the present invention (for example, DNA comprising the nucleotide sequence of Rad50) into a suitable expression vector, introducing the vector into a host cell, allowing the resulting transformant to express the polypeptide, and recovering the expressed polypeptide.

The variant polypeptide can be prepared, for example, by inserting a mutation into the amino acid sequence encoded by Rad50 polynucleotide, by a known method such as the PCR-mediated, site-directed-mutation-induction system (GIBCO-BRL, Gaithersburg, Md.), oligonucleotide-mediated, sight-directed-mutagenesis (Kramer, W. and Fritz, H J (1987) Methods in Enzymol. 154:350-367).

Methods of Formulation: The compounds described herein can be incorporated into pharmaceutical compositions. Such compositions typically include the active ingredient and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from pressured container or dispenser which contains a suitable propellant, e.g., a gas such as carbon dioxide, or a nebulizer. Such methods include those described in U.S. Pat. No. 6,468,798. Compositions for inhalation can also include propellants, surfactants, and other additives, e.g., to improve dispersion, flow, and bioavailability.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Compounds comprising nucleic acids e.g. Rad50, Smg6, Mre11, Nbs1, or the Mre11/Rad50/Nbs1 complex, fragments, mutants, variants and complementary sequences thereof, can also be administered by transfection or infection using methods known in the art, including but not limited to the methods described in McCaffrey et al. (2002), Nature, 418(6893), 38-9 (hydrodynamic transfection); Xia et al. (2002), Nature Biotechnol., 20(10), 1006-10 (viral-mediated delivery); or Putnam (1996), Am. J. Health Syst. Pharm., 53(2), 151-160, erratum at Am. J. Health Syst. Pharm., 53(3), 325 (1996). Compounds comprising nucleic acids can also be administered by method suitable for administration of DNA vaccines. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88(2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the compounds are prepared with carriers that will protect the active ingredient against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In some embodiments, the compounds (e.g., polypeptides) are modified to enhance delivery into cells, e.g., by the addition of an optimized or native TAT protein transduction domain (PTD), e.g., as described in Ho et al., Cancer Res. 61(2):474-7 (2001). Where the compound is a polypeptide, the polypeptide can be a fusion protein comprising an active portion (e.g., an active fragment of Apoptin) and a TAT PTD fused in frame.

The pharmaceutical compositions can be included in a container, pack, or dispenser together with instructions for administration.

Methods of Treatment: As used herein, the term “treatment” is defined as the application or administration of a therapeutic agent described herein, or identified by a method described herein, to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

Examples of routes of administration include parenteral, e.g., intravenous, intradermal, intracardial, intraperitoneal, subcutaneous, oral (e.g., inhalation), transdermal (topical), transmucosal, and rectal administration.

Therapeutic agents include, for example, proteins, nucleic acids, small molecules, peptides, antibodies, siRNAs, ribozymes, and antisense oligonucleotides. Dosage, toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a compound (i.e., an effective dosage) means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

Without further elaboration, it is believed that one skilled in the art can, using the preceding description, utilize the present invention to its fullest extent. The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification.

All publications and patent documents cited in this application are incorporated by reference in pertinent part for all purposes to the same extent as if each individual publication or patent document were so individually denoted. By their citation of various references in this document, Applicants do not admit any particular reference is “prior art” to their invention.

### EXAMPLES

Embodiments of inventive compositions and methods are illustrated in the following examples. These examples are provided for illustrative purposes and are not considered limitations on the scope of inventive compositions and methods. It will be appreciated that variations in proportions and alternatives in elements of the components shown will be apparent to those skilled in the art and are within the scope of embodiments of the present invention. Theoretical aspects are presented with the understanding that Applicants do not seek to be bound by the theory presented.

### Example 1

### Gene Expression in Patients

To identify genes that are overexpressed in patients with successful recovery of heart function, biopsies from patients with idiopathic cardiomyopathy which have been collected at the Johns Hopkins Hospital between 1997-2004 and stored in liquid nitrogen, were analyzed. A review of the biorepository biopsy samples were chosen from 25 patients with good prognosis and 18 patients with bad prognosis. Bad prognosis was defined as occurrence of death or an intervention, namely left-ventricular assist device placement or cardiac transplant, in the first 2 years after diagnosis, while good prognosis was defined as event free survival for greater than 5 years after diagnosis. The MIAME (Minimum information about a microarray experiment) guidelines were followed for all experiments. The tissue was homogenized with the MM301 instrument from Retsch and total RNA was isolated with the Micro-to-Midi Total RNA purification system from Invitrogen. Microarray analysis of total RNA was performed with the Human Genome U133 Plus 2.0 Array from Affymetrix. In all samples, both RNA isolation and microarray hybridization metal indices of quality control as specified in the Affymetrix Guideline for Assessing Sample and Array Quality. Raw expression values of all microarray chips were preprocessed with Robust Multiarray Average (RMA) in R.

In order to find differentially expressed genes between patients with good prognosis or bad prognosis, Significance Analysis of Microarrays (SAM) was performed. SAM defines significance of genes with the q-value, which is an adjusted p-value for multiple comparisons. A threshold of q<5% and FC>1.2 was used to identify significant differentially expressed genes. This approach resulted in 46 overexpressed genes in patients with a good clinical outcome relative to patients with a poor prognosis. Within this subset, two genes, SMG6 and RAD50, have been shown to play a key role in telomere maintenance and DNA repair. RAD50 has been shown to act through the Mre11/Rad50/Nbs1 (MRN) complex, which generates t-loops at human telomere ends. These t-loops prevent chromosome ends from being recognized as damaged DNA and provide a template for telomerase, with consequent preservation of genome stability. This increase in telomerase activity might explain a protective effect against degenerative processes and aging in the heart of patients with good prognosis. Without wishing to be bound by theory, it was hypothesized that the reason for better recovery in those individuals with good prognosis was caused by an increased capacity of their stem cell niche. This idea was further supported by experiments, showing that Rad50 played a role in the viability of stem cells.

Results: SAM analysis was performed on 30 samples to identify the most differentially expressed genes between patients with good prognosis and patients with bad prognosis (q<5%, FC>1.2). Among upregulated genes in patients with a good prognosis, RAD 50 and SMG 6, which both have key functions in telomerase activity were identified.

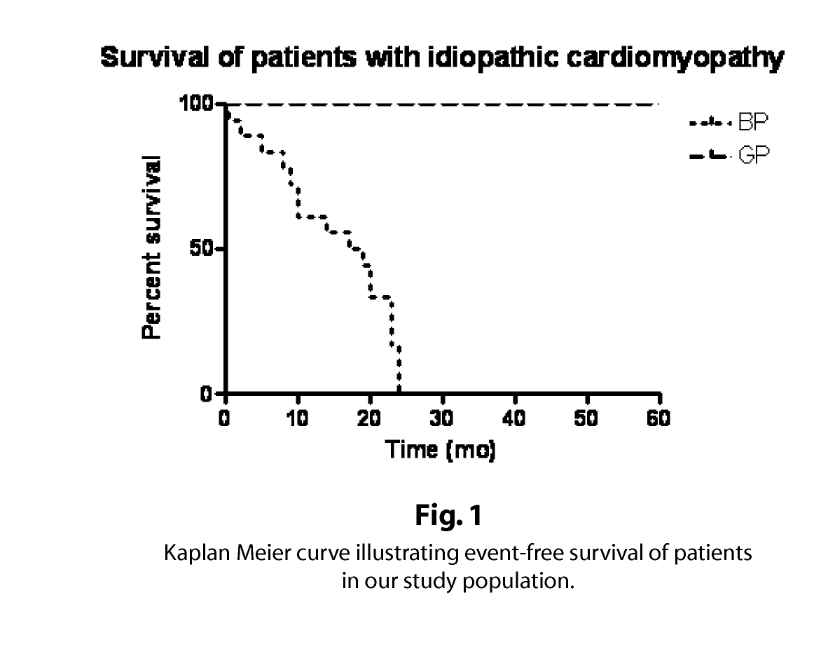

FIG. 1 is a Kaplan Meier curve illustrating event-free survival of patients in study population: While patients with a poor prognosis (BP) experienced an event (death, insertion of LVAD or cardiac transplant) within the first 2 years after presentation of heart failure, patients with good prognosis (GP) survived more than 5 years without any supporting device.

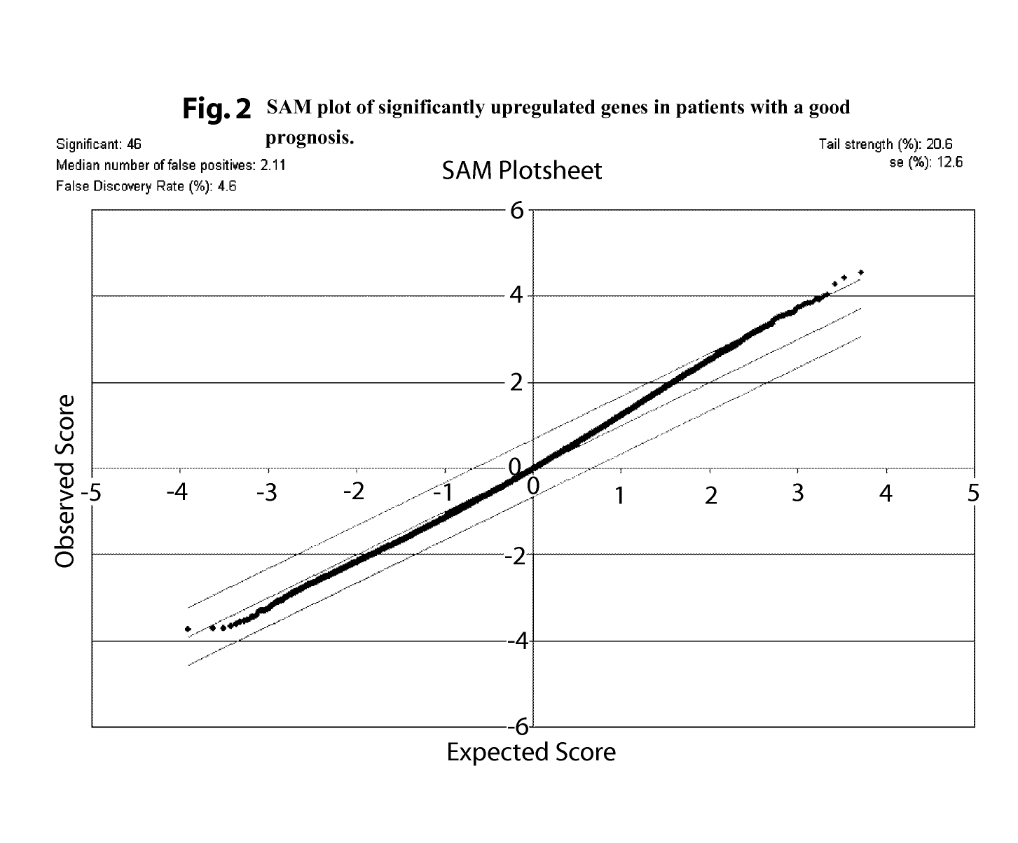

FIG. 2 is a SAM plot of significantly upregulated genes in patients with a good prognosis: a) 46 significantly upregulated genes in samples from patients with GP vs. BP (q<5%, FC>1.2, n=30).

Among over-expressed genes in patients with a good prognostic outcome, 2 genes, RAD50 and SMG6, were discovered which are regulators in telomerase activity and DNA repair, and which may potentially be involved in tissue regeneration. RAD50 is part of the Mre11/Rad50/Nbs1 (MRN) complex, a functional unit that generates t-loops at human telomere ends (Luo G, et al. Proc Natl Acad Sci USA. 1999; 96:7376-7381). These t-loops prevent chromosome ends from being recognized as damaged DNA and provide a template for telomerase, with consequent preservation of genome stability. This increase in telomerase activity may explain a protective effect against degenerative processes and aging in the heart of patients with good prognosis. Without wishing to be bound by theory, it can be speculated that the reason for better recovery in those individuals with good prognosis was caused by increased capacity of their stem cell niche. This idea was supported by these experiments, showing that Rad50 plays a role in the viability of stem cells.

Summary: The data provided here are the first demonstration that this pathway is upregulated in patients who recover with cardiomyopathy compared to those who do not and succumb to their disease. As such SMG6 and RAD50 may represent novel therapeutic targets which could be manipulated by gene therapy or small molecule strategies. Furthermore, since these pathways are important in stem cell viability, modifying stem cells to overexpress these pathways could enhance stem cell effectiveness for tissue repair.

### Example 2

### Gene Targets in Anti-Aging Therapy and Tissue Repair

To test whether Rad50 expression is affected by injury of the heart and consequently undergoes changes after myocardial infarction both in the tissue as well as in circulating blood cells.

Methods: Yorkshire pigs (n=6) were infarcted with balloon occlusion of a distal branch of the left anterior descending artery, tissue was harvested at the time of sacrifice 3 weeks post myocardial infarction (MI) and flash frozen in liquid nitrogen. Tissue samples included infarct zone (IZ), border zone (BZ) and remote zone (RZ), n=4 ea. In addition, blood samples were collected from all pigs (n=6), before and 20 minutes post MI, using PaxGene RNA tubes. Extraction of total RNA was performed with Trizol Standard Protocol and Invitrogen Purification kit for tissue samples, and the PaxGene RNA extraction kit for blood samples. Finally, total RNA was processed into cDNA (High capacity cDNA reverse transcription kit from Ambion), which was then used for quantification of Rad50 expression via realtime reverse transcriptase polymerase chain reaction. Taqman primers (Ambion) were used for RAD50 and 18S RNA as a housekeeping gene for normalization.

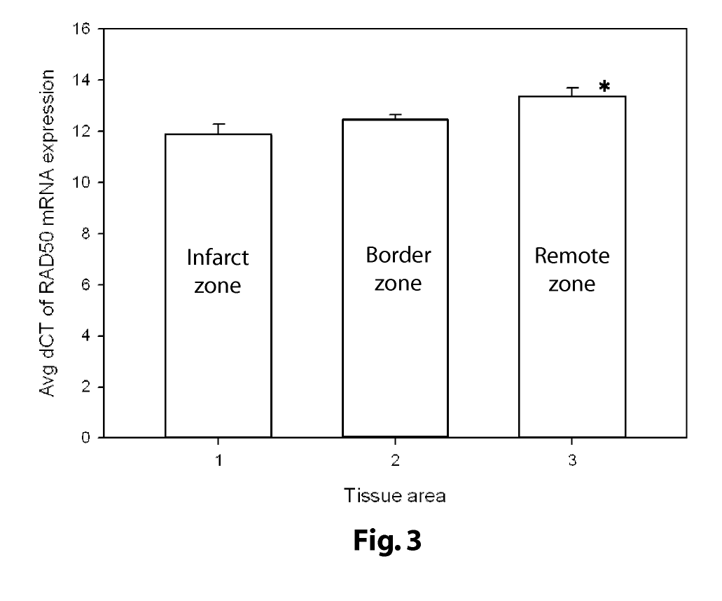

Results and Discussion: FIG. 3 illustrates average delta CT (avg dCT) values for Rad50 mRNA expression levels in different areas of the infarcted heart. This value is inversely related to the abundance of mRNA, therefore a high avg dCT value corresponds to a low amount of mRNA. RAD50 was overexpressed in the infarct zone (IZ) vs remote zone (RZ) (FC=2.8, P=0.045), however there was no difference in RAD50 expression levels between BZ and RZ or BZ and IZ. This data evidences that RAD50 is activated in the area of ischemic injury and may play a major role in regeneration of the heart.

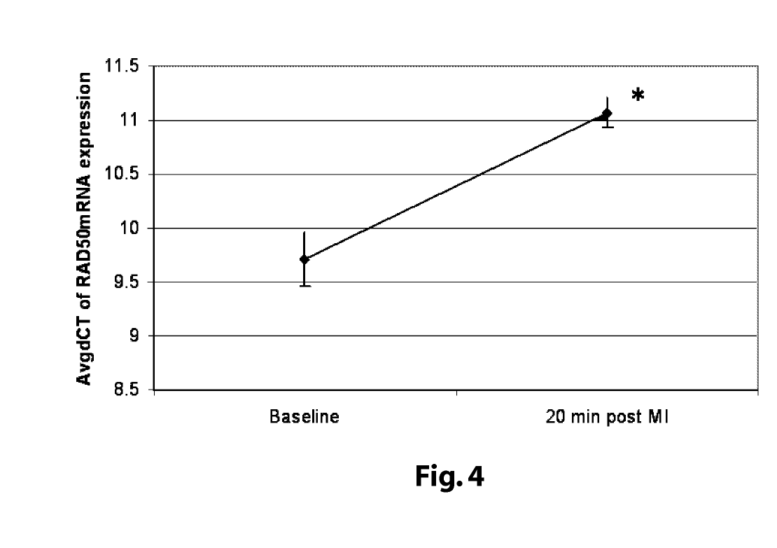

FIG. 4 depicts RAD50 expression levels in leukocytes before and after myocardial infarction. RAD50 expression in leukocytes was drastically reduced 20 minutes after myocardial infarction (FC=2.4, P=0.04). Without wishing to be bound by theory, recruitment of RAD50 expressing cells (possibly stem cells) into the injured myocardium, reflected as an acute decrease of RAD50 expression levels in the circulating blood cells.

Using realtime reverse transcriptase PCR, Rad50 expression was detected in both pig and human mesenchymal stem cells.

Summary of findings: In summary, it was shown that Rad50 expression levels were strongly involved in response to ischemic injury both in heart tissue as well as circulating blood cells. These observations strongly support our hypothesis that RAD50 plays an important regenerative role in patients with heart failure and is a promising therapeutic target, to support DNA repair, preserve genome stability and enhance regenerative capacity of stem cells and other types of cells that are involved in remodeling and repair mechanisms.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The Abstract of the Disclosure is provided to allow the reader to quickly ascertain the nature of the technical disclosure. It is submitted with the understanding that it will not be used to interpret or limit the scope or meaning of the following claims.

What is claimed is:

1. A biomarker composition prognostic for the long term survival of patients recovering from heart failure and cardiac diseases comprising Rad50 and Smg6 markers and polynucleotide and polypeptide molecules thereof.

2. The biomarker composition of claim 1, wherein the biomarker further comprises a marker selected from the group consisting of Mre11, Nbs1, Mre11/Rad50/Nbs1 complex, enzymes and t-loop generation factors.

3. The biomarker composition of claim 2, wherein the biomarker comprises the Rad50, Smg6 and Mre11 markers and at least one marker comprising a polynucleotide, fragments, alleles, variants, mutants, complementary sequences thereof of Rad50, Smg6 and Mre11.

4. The biomarker composition of claim 2, wherein the biomarker further comprises the Rad50, Smg6 and Mre11 markers and at least one marker comprising a polypeptide, fragment, variants, analogs and mutants thereof of Rad50, Smg6 and Mre11 or combinations thereof.

5. The biomarker composition of claim 3, wherein the biomarker further comprises the Rad50, Smg6 and Mre11 markers and at least one marker comprising a polynucleotide, fragments, alleles, variants, mutants substituted polynucleotides and complementary sequences thereof of Rad50.

6. The biomarker composition of claim 4, wherein the biomarker further comprises the Smg6, Rad50 and Mre11 markers and at least one marker comprising a polypeptide, fragment, variants, analogs and mutants thereof of Rad50.

7. The biomarker composition of claim 3, wherein the biomarker further comprises the Smg6, Rad50 and Mre11 markers and at least one marker comprising a polynucleotide, fragments, alleles, variants, mutants, substituted polynucleotides and complementary sequences thereof of Smg6.

8. The biomarker composition of claim 4, wherein the biomarker further comprises the Smg6, Rad50 and Mre11 markers and at least one marker comprising a polypeptide, fragment, variants, analogs and mutants thereof of Smg6.

9. A biomarker composition for the diagnosis of heart failure and cardiac diseases comprising markers Rad50, Smg6 and Mre11.

10. The biomarker composition of claim 9, wherein the biomarker further comprises a marker selected from the group consisting of Mre11, Nbs1, Mre11/Rad50/Nbs1 complex, enzymes and t-loop generation factors.

11. The biomarker composition of claim 9, wherein the biomarker comprises the Rad50, Smg6 and Mre11 markers and at least one marker comprising a polynucleotide, fragments, alleles, variants, mutants, complementary sequences thereof of Rad50, Smg6 and Mre11.

12. The biomarker composition of claim 9, wherein the biomarker further comprises the Rad50, Smg6 and Mre11 markers and at least one marker comprising a polypeptide, fragment, variants, analogs and mutants thereof of Rad50, Smg6 and Mre11.

13. The biomarker composition of claim 9, wherein the biomarker further comprises the Rad50, Smg6 and Mre11 markers and at least one marker comprising a polynucleotide, fragments, alleles, variants, mutants substituted polynucleotides and complementary sequences thereof of Rad50.

14. The biomarker composition of claim 9, wherein the biomarker further comprises the Rad50, Smg6 and Mre11 markers and at least one marker comprising a polypeptide, fragment, variants, analogs and mutants thereof of Rad50.

15. The biomarker composition of claim 9, wherein the biomarker further comprises the Rad50, Smg6 and Mre11 markers and at least one marker comprising a polynucleotide, fragments, alleles, variants, mutants, substituted polynucleotides and complementary sequences thereof of Smg6.

16. The biomarker composition of claim 9, wherein the biomarker further comprises the Rad50, Smg6 and Mre11 markers and at least one marker comprising a polypeptide, fragment, variants, analogs and mutants thereof of Smg6.

17. An agent that specifically binds a biomarker composition comprising Rad50, Smg6 and Mre11, Mre11/Rad50/Nbs1 complex, enzymes and t-loop generation factors, variants, mutants, and fragments thereof.

18. The agent of claim 17, wherein said agent comprising an antibody, aptamer or complementary polynucleotides which hybridize under stringent hybridization conditions to Rad50, Smg6 and Mre11, the Mre11/Rad50/Nbs1 complex, enzymes and t-loop generation factors, variants, mutants, and fragments thereof.

19. An isolated cell expressing Smg6 and/or Rad50 and/or Mre11/Rad50/Nbs1 complex, enzymes and t-loop generation factors, variants, mutants, and fragments thereof.

20. The isolated cell of claim 19, wherein the cell is a mammalian cell.

21. The isolated cell of claim 19, wherein the cell comprises a stem cell, monocyte, lymphocyte, neutrophil, eosinophil, mast cell, natural killer cells, myocytes, heart cells, and fibroblasts.

22. The isolated cell of claim 19, wherein the cell comprises a vector expressing Smg6 and/or Rad50 and/or Mre11, and/or Mre11/Rad50/Nbs1 complex, enzymes and t-loop generation factors, variants, mutants, and fragments thereof.

23. The cell of claim 22, wherein Smg6, Rad50, Mre11, Mre11/Rad50/Nbs1 complex, enzymes and t-loop generation factors variants, mutants and fragments thereof are operatively linked to a tissue specific promoter, a constitutive or inducible promoter.

24. A vector expressing Smg6 and/or Rad50, and/or Mre11, and/or Mre11/Rad50/Nbs1 complex, enzymes and t-loop generation factors, variants, mutants, and fragments thereof.

25. The vector of claim 24, wherein the Smg6 and/or Rad50, and/or Mre11, Mre11/Rad50/Nbs1 complex are operably linked to a tissue-specific promoter, a constitutive or inducible promoter.

26. The vector of claim 24, wherein the vector expresses Smg6, Rad50 and Mre11 variants, mutants, alleles, and fragments thereof.

27. A method of treating heart failure comprising:
administering to a patient a composition comprising Rad50, Smg6 and Mre11 or Mre11/Rad50/Nbs1 complex, enzymes and t-loop generation factors, variants, mutants, and fragments thereof; and,
treating heart failure.

28. The method of claim 27, wherein the Rad50, Smg6 and Mre11 and the Mre11/Rad50/Nbs1 complex comprise polynucleotides and/or polypeptides.

29. The method of claim 27, wherein the composition comprises an expression vector encoding Rad50, Smg6 and Mre11 or the Mre11/Rad50/Nbs1 complex operably linked to a promoter.

30. The method of claim 29, wherein the promoter comprises a tissue specific promoter, a constitutive tissue promoter or an inducible tissue specific promoter.

31. The method of claim 27, wherein cells are treated with an agent which increases expression of Smg6 and/or Rad50 and/or Mre11.

32. A method of treating heart failure comprising:
isolating cells from a patient or donor;
administering to the cells a composition comprising Rad50, Smg6 and Mre11 or Mre11/Rad50/Nbs1 complex, enzymes and t-loop generation factors, variants, mutants, and fragments thereof;
culturing the cells ex-vivo;
administering the cells to a patient; and,
treating heart failure.

33. The method of claim 32, wherein the cells over-express Rad50, Smg6 and Mre11, Mre11/Rad50/Nbs1 complex, enzymes and t-loop generation factors by at least about 10% as compared to a normal cell.

34. The method of claim 32, wherein the cells over-express Rad50, Smg6 and Mre11.

35. The method of claim 32, wherein the cells over-express Smg6.

36. The method of claim 32, wherein the cells over-express Rad50.

37. The method of claim 32, wherein the cells over-express at least one polynucleotide and/or polypeptide from the Mre11/Rad50/Nbs1 complex, enzymes and t-loop generation factors.

38. The method of claim 32 wherein the cells are autologous, heterologous, syngeneic, or allogeneic cells.

39. The method of claim 32, wherein the cells comprising: stem cell, monocyte, lymphocyte, neutrophil, eosinophil, mast cell, natural killer cells, myocytes, heart cells, fibroblasts.

40. A method of treating myocardial infarction and cardiac disorders comprising:
isolating stem cells from a patient or donor;
transforming the isolated stem cells with a vector expressing Rad50, Smg6 and Mre11, and/or Mre11/Rad50/Nbs1 complex, enzymes and t-loop generation factors, variants, mutants, and fragments thereof, as compared to a normal cell, and/or incubating the stem cells with a pharmaceutical agent which increases the expression of Smg6 and/or Rad50, and/or Mre11 variants, mutants, and fragments thereof as compared to a normal cell;
administering the cells to a patient; and,
treating myocardial infarction and cardiac disorders.

41. A method of treating heart failure comprising:
administering to a patient a pharmaceutical composition comprising nucleic acids and/or peptides of Rad50, Smg6 and Mre11, or Mre11/Rad50/Nbs1 complex, enzymes and t-loop generation factors, variants, mutants, and fragments thereof; and,
treating heart failure.

42. A method of treating myocardial infarction and cardiac disorders comprising:
administering to a patient a pharmaceutical composition comprising nucleic acids and/or peptides of Rad50, Smg6, Mre11, or Mre11/Rad50/Nbs1 complex, enzymes and t-loop generation factors variants, mutants, and fragments thereof; and,
treating myocardial infarction and cardiac disorders.

43. The method of 42, wherein Rad50, Smg6, Mre11, Mre11/Rad50/Nbs1 complex, enzymes and t-loop generation factors are upregulated as compared to a control.

44. A method of selecting cells to treat a patient in need of stem cell implantation comprising: isolating a stem cell; screening the stem cell for expression of at least one of Rad50, Smg6, or Mre11; separating the Rad50, Smg6 and Mre11 expressing stem cells; and, selecting said stem cells to treat a patient in need of stem cell implantation.

45. The method of claim 44, wherein said stem cell is autologous or donor-derived.

46. The method of claim 44, wherein expression of Rad50, Smg6 and Mre11 comprises polynucleotides and polypeptides.

47. The method of claim 44, wherein the stem cells are cultured with agents which increase expression of at least one of Rad50, Smg6 and Mre11.

48. The method of claim 44, wherein a vector expressing Rad50, Smg6 or Mre11 is administered to the stem cells.

49. A pharmaceutical composition comprising nucleic acids and/or peptides of Rad50, Smg6 and Mre11, variants, mutants, and fragments thereof in a therapeutically effective concentration.

50. A method of identifying candidate agents for modulating expression of Rad50, Smg6 or Mre11 comprising:
providing a biological sample;
incubating the biological sample with a candidate agent;
screening the biological sample for expression of Rad50, Smg6, Mre11, Mre11/Rad50/Nbs1 complex, enzymes or factors involved in the generation of t-loops;
comparing the expression levels between the biological sample and control; and,
identifying an inhibitor.

51. The method of claim 50, wherein the Rad50, Smg6 and Mre11 expression levels are upregulated in the biological sample contacted with a candidate agent as compared to a control sample.

52. The method of claim 50, wherein Smg6 expression levels are determined by measuring the amount of at least one of Smg6 polypeptide, Smg6 mRNA, and Smg6 cDNA.

53. The method of claim 50, wherein the expression levels of Rad50 are determined by measuring the amount of at least one of Rad50 polypeptide, Rad50 mRNA, and Rad50 cDNA.

54. The method of claim 50, wherein the expression levels of Mre11 are determined by measuring the amount of at least one of Mre11 polypeptide, Mre11 mRNA, and Mre11 cDNA.

55. The method of claim 50, wherein the expression levels of Smg6, Rad50 and Mre11 are determined by detecting the amount of a transcribed polynucleotide or portion thereof.

56. The method of claim 55, wherein the transcribed polynucleotide is an mRNA.

57. The method of claim 55, wherein the transcribed polynucleotide is a cDNA.

58. The method of claim 55, wherein the detecting further comprises amplifying the transcribed polynucleotide or portion thereof.

59. A method of identifying candidate therapeutic agents for modulating expression of Rad50, Smg6 and Mre11 comprising:
providing a biological sample;
incubating the biological sample with a candidate therapeutic agent;
screening the biological sample for expression of Rad50, Smg6 and Mre11 and Mre11/Rad50/Nbs1 complex, enzymes or factors involved in the generation of t-loops;
comparing the expression levels between the biological sample and control; and,
identifying a candidate therapeutic agent.

60. The method of claim 59, wherein the biological sample comprises fluid, a cell, tissues, protein, peptides, amino acids, and nucleic acids.

61. The method of claim 59, wherein the Rad50, Smg6, Mre11 and Mre11/Rad50/Nbs1 complex, enzymes or factors involved in the generation of t-loops are detected using an immunoassay, SDS-PAGE gel, blotting, or biochip.

62. The method of claim 59, wherein the Rad50, Smg6, Mre11 and/or Mre11/Rad50/Nbs1 complex, enzymes or factors involved in the generation of t-loops are detected using a biochip array.

63. The method of claim 62, wherein the biochip array is a protein chip array.

64. The method of claim 62, wherein the biochip array is a nucleic acid array.

65. A method of treating heart failure comprising:
isolating cells from a patient or donor;
screening the cells for expression of Rad50, Smg6, Mre11 or Mre11/Rad50/Nbs1 complex, enzymes and t-loop generation factors, variants, mutants, and fragments thereof;
culturing the cells ex-vivo;
administering the cells to a patient; and,
treating heart failure.

66. The method of claim 65, wherein the cells express Rad50, Smg6 and Mre11.

67. The method of claim 65, wherein the cells express at least one polynucleotide and/or polypeptide from the Mre11/Rad50/Nbs1 complex, enzymes and t-loop generation factors.

68. The method of claim 65 wherein the cells are autologous, heterologous, syngeneic, or allogeneic cells.

69. The method of claim 65, wherein the step of culturing the cells further comprising administering agents which increase expression of at least one of Rad50, Smg6 or Mre11.

70. A method of treating and reducing cardiovascular aging comprising administering to a patient a pharmaceutical composition comprising nucleic acids and/or peptides of Rad50, Smg6 and Mre11, variants, mutants, and fragments thereof, and/or cells comprising Rad50, Smg6 and Mre11, variants, mutants, and fragments thereof; and,
treating and reducing cardiovascular aging.

71. A method of repairing and maintaining telomeres and modulating telomere activity comprising administering to a cell nucleic acids and/or peptides comprising Smg6, Rad50, Mre11, variants, mutants, and fragments thereof, and/or cells comprising Smg6, Rad50, Mre11, variants, mutants, and fragments thereof expressing; and,
repairing and maintaining telomeres and modulating telomere activity.

72. A kit comprising a biomarker comprising at least one of: Rad50, Smg6, Mre11, Nbs1, or Mre11/Rad50/Nbs1 complex.

73. The kit of claim 72, wherein said kit further comprises an agent for detection of at least one of Rad50, Smg6, Mre11, Nbs1, or the Mre11/Rad50/Nbs1 complex.

74. The kit of claim 73, wherein said agent comprising an antibody, aptamer, oligonucleotide probe, polynucleotide, or polypeptide.


SEQUENCE LISTING (2 sequences)

SEQ ID NO: 1 - protein, 6 aa, Artificial Sequence
  Description of Artificial Sequence Synthetic 6xHis tag
HHHHHH

SEQ ID NO: 2 - protein, 10 aa, Artificial Sequence
  Description of Artificial Sequence Synthetic 10xHis tag
HHHHHHHHHH